(19) United States
(12) Patent Application Publication
(10) Pub. No.: US 2005/0154537 A1
(43) Pub. Date: Jul. 14, 2005

(54) Methods, program products, and systems for single and multi-agent dosing and other related methods

(76) Inventors: John D. Kutzko, Nokomis, FL (US); Michaeal G. Singer, Harrisville, MI (US)

Correspondence Address: BRACEWELL & PATTERSON, LLP;IP DOCKETING, P.O. BOX 61389, HOUSTON, TX, 77208-1389, US

(21) Appl. No.: 11/009,271

(22) Filed: Dec. 10, 2004

Related U.S. Application Data
(60) Provisional application No. 60/529,385, filed on Dec. 12, 2003.

(51) Int. Cl. G06F 17/60; G06F 19/00; G01N 33/48; G01N 33/50; A61K 31/44; A01N 43/40

(52) U.S. Cl. 702/19; 705/2

(57) ABSTRACT

Methods, program product, and systems for single and multi-agent dosing are provided. An embodiment of a method for revising a cycle dose of at least one agent for a patient receiving a single or multi-agent therapy includes accepting as a first input the patient's cycle dose(s) of the at least one agent, accepting as a second input determination of the relevancy of at least one surrogate marker indicating a pharmacologic response of the patient and determining a revised dose of the at least one agent. Embodiments of systems and program products are also provided for use in calculating revised dose of at least one agent.

### RELATED APPLICATIONS

This application is related to and claims priority and benefit of U.S. Provisional Patent Application Ser. No. 60/529,385, filed Dec. 12, 2003, titled “Methods, Software and Systems for Single and Multi-Agent Dosing and Other Related Methods,” which is incorporated herein by reference in its entirety.

### BACKGROUND OF THE INVENTION

1. Field of the Invention

The present invention relates in general to the field of pharmacology and pharmacotherapy. More particularly, the present invention relates to methods, program products, and systems to modify drug dose to enhance therapeutic and other treatments and manage adverse drug effects as related to the response of surrogate markers for single and multi-agent dosing of drugs to benefit patient care and drug development.

2. Description of Related Art

Pharmaceutical agents are developed in accordance to established regulatory management and are required to demonstrate safety and efficacy in homogeneous populations. After approval they are applied to heterogeneous patient populations in which to improve individual effectiveness dose modification techniques have evolved from the gestalt of the prescriber to pharmacologic sciences tracking the evolution of computer technology.

The balance between optimized therapeutic effect and acceptable degrees of adverse side effects is based upon the properties of the agent and the milieu of ‘Interindividual Variability’ presented by each unique patient.

Initial forays into managing this ‘balance’ were focused on laboratory monitoring of the time, distribution and concentration of the agent in the patient monitoring its therapeutic effect and correlating outcome to measured values. Therapeutic drug monitoring met with success over ‘gestalt’ as increased scrutiny and prevention of overt excursions to extremes improved outcomes somewhat, but the cost, time, logistics and availability of such systems are limited and the parameters utilized are not routine in clinical practice. Other methods were developed, tried and met with limited effectiveness; Bayesian, Pharmacokinetics-Pharmacodynamics, Population Sampling and eventually so-called; ‘Expert Systems’. These methods were based upon historical response and projected responses that provided only incremental improvement. Bridging the increasing gap of individualized dosing, however optimal therapeutic effect and acceptable economic remained unsolved.

As computer and medical technologies became available and evolved beginning in the 1970's and continuing to this date; ‘Expert Systems’ and new ‘Dosing Schemes’ art were published utilizing the sheer power of computer management of vast volumes of data to predict outcome. Initially in medical histories (U.S. Pat. No. 4,130,881), treatment protocols (U.S. Pat. No. 4,839,822) and eventually drug dosing (U.S. Pat. Nos. 4,880,014 and 5,019,874). Each new attempt based upon narrow spectra diseases demonstrated minimal improvement, used complex and/or specialized systems and variables.

Recent art has demonstrated improved effectiveness but focused on single agents, doses and fixed blood values (U.S. Pat. Nos. 5,365,948, 5,542,436, and 5,694,950) providing even more narrow and limited practical use.

Recent art, some co-authored by the current Applicants, has evolved to multiple agents and markers, but Applicants have recognized that this art failed to recognize the cumulative drug effect of the usual and customary clinical cycle of drug therapy and the critical aspect of the validity of the decision based data of the surrogate marker used to base dose modifications on; (U.S. Pat. Nos. 6,578,582, 6,575,169, 6,267,116, 6,581,606, and 6,581,607).

### SUMMARY OF THE INVENTION

In view of the foregoing, recognizing the limits of their previous efforts the current Applicants have developed embodiments of the present invention to accommodate a complete ‘Cycle’ of drug therapy, the validity of the markers and the need for variable response to fit the variable dynamic presentation of the Individual characteristics of the patient. In addition, embodiments of the present invention have added the classification of ‘non-pharmacologic’ surrogate markers in recognition of subjective prescriber assessment.

Applicants also recognized that there is a need for enhanced methods, program products, and systems to customize and individualize drug dosing for patients, which are based upon individual patient's response. There is also a need for methods, program products, and systems for use in treating a patient with one or more pharamcologic and non-pharmacological modalities to optimize pharmacologic outcome and reduce the risk of an adverse drug event while assessing the clinical relevancy and validity of a measured surrogate marker.

Embodiments of the present invention advantageously provide methods, program products, and systems to revise a dose of at least one agent in a therapy for a patient. An embodiment of a method of the present invention, for example, includes accepting as a first input the patient's cycle dose(s) of the at least one agent, accepting as a second input determination of the relevancy of at least one surrogate marker indicating a pharmacologic response of the patient and determining a revised cycle dose of the at least one agent. The revised dose can be determined as a function of the first input cycle dose(s) and second input determination of the relevancy of the at least one non-pharmacologic modality.

Another embodiment of a method of the present invention advantageously provides a method for revising a dose of at least one agent in a therapy for a patient receiving such a therapy. This method, for example, includes accepting as a first input the patient's cycle dose(s) of the at least one agent, accepting as a second input at least one non-pharmacologic modality received by the patient, accepting as a third input determination of the relevancy of at least one surrogate marker indicating a pharmacologic response of the patient, accepting as a fourth input determination of the relevancy of the at least one non-pharmacologic modality indicating achievement of a benefit and determining a revised cycle dose of the at least one agent.

Advantageously, the revised dose can be determined as a function of the cycle dose(s), the at least one non-pharmacologic modality, determination of the relevancy of the at least one surrogate markers and determination of the relevancy of the at least one non-pharmacologic modality. For example, such calculation of the revised dose can be based on the cumulative effect of the at least one agent and the at least one non-pharmacologic modality to be achieved by the single or multi-agent therapy and non-pharmacologic intervention.

Another embodiment of the present invention advantageously provides a system for revising a cycle dose of at least one agent in a therapy for a patient receiving such a therapy. A system, for example, includes means for accepting as a first input the patient's cycle dose(s) of the at least one agent, means for accepting as a second input determination of the relevancy of at least one surrogate marker indicating a pharmacologic response of the patient, and means responsive to the first input and second input for calculating a revised cycle dose of the at least one agent.

Yet another embodiment of the present invention advantageously provides a system for revising a cycle dose of at least one agent in a therapy for a patient. This embodiment of a system includes means for accepting as a first input the patient's cycle dose(s) of the at least one agent, means for accepting as a second input at least one non-pharmacologic modality received by the patient, means for accepting as a third input determination of the relevancy of at least one surrogate marker indicating a pharmacologic response of the patient, means for accepting as a fourth input determination of the relevancy of the at least one non-pharmacologic modality indicating achievement of benefit, and means for determining a revised cycle dose of the at least one agent as a function of the first input cycle dose(s), the at least one non-pharmacologic modality, determination of the relevancy of the at least one surrogate markers and determination of the relevancy of the at least one non-pharmacologic modality.

Still another embodiment of the present invention advantageously provides a program product or software to revise a cycle dose of at least one agent in a therapy for a patient receiving such a therapy. This program product includes means for accepting as a first input the patient's cycle dose(s) of the at least one agent, means for accepting as a second input determination of the relevancy of at least one surrogate marker indicating a pharmacologic response of the patient, and means responsive to the first input and second input for calculating a revised cycle dose of the at least one agent.

Still yet another embodiment of the present invention advantageously provides a program product or software to revise a cycle dose of at least one agent in a therapy for a patient receiving such a therapy. This program product, for example, includes means for accepting as a first input the patient's cycle dose(s) of the at least one agent, means for accepting as a second input at least one non-pharmacologic modality received by the patient, means for accepting as a third input determination of the relevancy of at least one surrogate marker indicating a pharmacologic response of the patient, means for accepting as a fourth input determination of the relevancy of the at least one non-pharmacologic modality indicating achievement of benefit, and means for determining a revised cycle dose of the at least one agent as a function of the first input cycle dose(s), the at least one non-pharmacologic modality, determination of the relevancy of the at least one surrogate markers and determination of the relevancy of the at least one non-pharmacologic modality.

Yet another embodiment of the present invention advantageously provides a program product or software to revise a cycle dose of at least one agent in a therapy for a patient receiving such a therapy. This program product, for example, includes a first receiver to accept as a first input the patient's cycle dose(s) of the at least one agent, a second receiver to accept as a second input determination of the relevancy of at least one surrogate marker indicating a pharmacologic response of the patient, and a calculator to calculate a revised cycle dose of the at least one agent.

Yet another embodiment of the present invention advantageously provides a program product or software to revise a cycle dose of at least one agent in a therapy for a patient receiving such a therapy. This program product advantageously includes a first receiver to accept as a first input the patient's cycle dose(s) of the at least one agent, a second receiver to accept as a second input at least one non-pharmacologic modality received by the patient, a third receiver to accept as a third input determination of the relevancy of at least one surrogate marker indicating a pharmacologic response of the patient, a fourth receiver to accept as a fourth input determination of the relevancy of the at least one non-pharmacologic modality indicating achievement of benefit, and a calculator to calculate a revised cycle dose of the at least one agent as a function of the first input cycle dose(s), the at least one non-pharmacologic modality, determination of the relevancy of the at least one surrogate markers and determination of the relevancy of the at least one non-pharmacologic modality.

Advantageously, embodiments of the methods, systems and program products of the present invention each focuses on a cycle dose, rather than a current dose as known in the art. Cycle dose is a summative or cumulative dose administered for a time period during which the maximum therapeutic effect of a single or multiple agent(s) is realized. Applicants have recognized that it is the agent's total amount over a time period based upon the specifics of the agent and the disorder is more accurate, appropriate and effective. The present invention considers doses administered during a selected period of time, which better accommodate the variability of compliance and the time needed for the agent to demonstrate its efficacy or toxicity. The cycle dose is also based upon how a prescriber actually practices, thinks and acts out the process.

Still advantageously, embodiments of the methods, systems and program products of the present invention each can analyze two or more surrogate markers concurrently rather than one marker at a time. This multi-marker analysis provides a real-time measurement of the cumulative affect of one or more pharmacologic agents and one or more non-pharmacologic modalities. Further advantageously, embodiments of the methods, systems and program products of the present invention can provide more accurate information on efficacy of drugs that have been shelved for a long time, which would help pharmaceutical companies to reclaim the drugs. Additionally, these embodiments are non-invasive, precise and sensitive.

### BRIEF DESCRIPTION OF THE DRAWINGS

Some of the features, advantages, and benefits of the present invention having been stated, others will become apparent as the description proceeds when taken in conjunction with the accompanying drawings, in which:

FIG. 1 is a flow chart of a process by which revised cycle doses of at least one agent are determined according to an embodiment of the present invention;

FIG. 2 is a flow chart of a process by which revised cycle doses of at least one agent are determined according to an embodiment of the present invention;

FIG. 3 demonstrates a process of receiving the data input from medical personnel of a single or multi-agent therapy according to an embodiment of the present invention;

FIG. 4 is a schematic diagram of a system and program product to calculate revised doses of at least one agent according an embodiment of to the present invention; and

FIG. 5 is a schematic diagram of a doser or apparatus to calculate revised doses of at least one agent according to another embodiment of the present invention.

### DETAILED DESCRIPTION

The present invention now will be described more fully hereinafter with reference to the accompanying drawings in which illustrated embodiment(s) of the invention are shown. This invention may, however, be embodied in many different forms and should not be construed as limited to the illustrated embodiments set forth herein; rather, the embodiments are provided so that this disclosure will be thorough and complete and will fully convey the scope of the invention to those skilled in the art. Like numbers refer to like elements throughout.

Presently drug dose determination is based upon population findings revealed in a regulatory approval process and not individualized to a patient's response. Methods to provide a customized or individual drug dose can benefit patient care and drug development. The actions of the drug and the response of the patient should each be considered. The dose response or fit of the agent can be more clearly understood and methods, systems, dosers, and program products to revise that dose based upon the individual patient's response can provide an additional dimension of drug delivery enhancement. When a drug is given and a response is established the relationship of that response in terms of initial dose to response to present dose provides a better understanding of the fit of the drug as it relates to the response. The response may be positive and efficacious or negative and toxic. A multi-dimensional illustration, whose plots include the current dose, the new dose planned and the percent change of the individual's response to the agent, for example, can be used. In this manner the effect of the new dose based upon the experience of the drug effects can be used to understand and predict the effect that the patient is under. Next the patient's individual experience in terms of responding as expected, less than expected or more than expected; from what ever circumstance is considered. In this manner the dose can be modified and increased, left the same or reduced to achieve a custom individualized optimal effect.

The amount of drug given over fixed period of time or cycle is considered so as to account for the cumulative effect the patient is under, as well as the maturity and therefore the relevancy of the marker of efficacy or toxicity used to interpret the response of the agent(s) and modify the dose(s) accordingly. In this manner the cycle can be revised or adjusted based on clinical practice, such as cycle-dosing in chemotherapy, route of administration (oral vs. intravenous) and duration and mechanism of action in multiple agents. In disease conditions requiring multiple pharmacologic interventions, with each intervention having additive or synergistic effects, the cycle can be modified to accommodate the different agents' route of administration and pharmacokinetic and pharmacodynamic characteristics.

Indicators or markers of efficacy or toxicity are based upon the disease state and the agents' pharmacologic profile. They are used to quantify the desired and undesired response to the agent(s) received as a measure of progress, effectiveness or qualified levels of toxicity. These indicators and subsequent changes are illustrated on the dose-response curve of that agent in this particular patient and are used to modify subsequent doses. For instance, a chemotherapeutic agent may induce an acceptable level of toxicity in which exposure of the agent is maximized and that level of toxicity experienced by the patient is accepted or a dose reduction is made and the toxicity experience is ameliorated or eliminated.

Each marker has its own dose-response curve. Marker response is concurrently characterized by a dynamic and on-going balance of positive and negative effect. The increase in dose to produce a desired effect may be accompanied by an attendant noxious effect that should be maintained within a margin of safety. By illustrating these positive and negative dynamics the prescriber can better manage the margin between optimal therapeutic effect and acceptable levels of toxicity. This dose modification technique allows the prescriber simultaneous access to markers of efficacy and toxicity enabling dose adjustment to be fully optimized while balancing and recognizing the agents' positive and negative effects.

The dose-response relationship is characterized as either inverse or direct, in that in some instances more agent may result in a decrease in the marker or less agent may result in a marker increase (inverse). In a direct relationship more agent increases the marker and less agent reduces the marker value.

To further individualize the dose adjustment to the unique character and needs of the patient a variable percentage amount of the dose is reserved based upon the pharmacologic properties of the drug and disease under management. This reserve fraction (Variable Sensitivity Value) is established for each agent and marker when the dose modifier is constructed.

To further refine the impact of non-pharmacologic dynamics (nutrition-exercise-activity) that directly impact the dose-marker-response relationship an Efficacy Toxicity Determinant Factor is provided. This value can be adjusted to accommodate the impact of non-pharmacologic effects such as grapefruit juice and the absorption of certain agents and exercise/activity in the management of diabetes.

Embodiments of methods, systems, dosers, and program products of the present invention can use numerous surrogate markers, including blood cell counts, viral load measurements and serum, plasma or whole blood levels of drug, to determine the best next required dose of each individual agent for a patient. Any objective or subjective measurement can be used as long as that surrogate marker(s) is reflective of the combined multiple agent's therapeutic efficacy or toxicity. The individual agent that bears the greater responsibility for the efficacious or toxic results reported for the combined pharmacological or non-pharmacological modality are identified. By identifying that agent, the dosing modification scenario can allow the prescriber to potentially intensify the dosage of the less offensive agent while reducing the dose of the offending agent by a specific amount, relative to the overall toxicity of the combination therapy.

Given the efficacious or toxic nature of each agent's pharmacologic mechanism of action, relative to the surrogate marker being measured, a change in each agent's drug dose can be determined, e.g., calculated, by the system, doser, or program product which uses a stochastic loop mechanism. Conversely, by employing these methods, systems, dosers, or program products, one can determine the expected result of each individual agent's drug dose change based on the surrogate markers. Each specific surrogate marker affected by the combined pharmacologic intervention is identified to scrutinize and determine if the reported measurement is reflective of the most recent dosing episode(s) of the multiple pharmacological or non-pharmacological modalities. There are known latencies that occur, with regard to a surrogate marker measurement, which may or may not accurately reflect the absolute measure of efficacy or toxicity relative to the characteristics of each agent in the combined pharmacological modality, depending on the correct timing of the surrogate marker value measured.

The term “pharmacologic intervention” as used herein means all biological substances and includes, but is not limited to, vaccines, serums, drugs, adjuvants to enhance or modulate a resulting immune response, vitamin antagonists, medications and all substances derived from and/or related to the foregoing substances.

The term “non-pharmacologic intervention” as used herein means diet, aerobic and anaerobic exercise and all other non-biological and non-pharmaceutical substances.

The term “agent” as used herein means all biological substances and includes, but is not limited to, vaccines, serums, drugs, adjuvants to enhance or modulate a resulting immune response, vitamin antagonists, medications and all substances derived from and/or related to the foregoing substances.

The term “surrogate marker” as used herein means all surrogate markers and includes, but is not limited to, a measurement of biological activity within the body which indirectly indicates the effect of treatment on a disease state or on any condition being treated; and any measurement taken on a patient which relates to the patient's response to an intervention, such as the intervention of a biological substance introduced into or on the patient.

The term “latency of a marker” as used herein means the time period between cumulative dose administration of both single and multiple agent(s) and full development of marker maturity. It is a function of the combined factors of the individual, pharmacologic properties of the agent(s) and disease process.

The term “relevancy of a marker” as used herein means objective quantification of the marker's maturity. A relevant marker is of such quality to support an optimal clinical decision.

The term “validity of a marker” as used herein means the time at which the surrogate marker is fully impacted by the effects of the drug(s). The valid surrogate marker illustrates the total effect of the drug(s) on patient.

The term “cycle” in cycle dose as used herein means the time period during which the maximum therapeutic effect of a single or multiple dose(s) of the agent(s) administered is realized.

The time periods for the occurrence of a therapeutic and toxic effect may differ among individuals. While awaiting the development of the optimal or significantly enhanced efficacious effect, a deleterious or adverse event may require interim monitoring, dose adjustment or adjuvant therapies.

An embodiment of the present invention advantageously provides a method 100 for providing a revised dose of at least one agent in a therapy for a patient receiving such a therapy. This method advantageously includes accepting as a first input the patient's cycle dose(s) of the at least one agent (block 101), accepting as a second input determination of the relevancy of at least one surrogate marker indicating a pharmacologic response of the patient (block 102) and determining a revised cycle dose of the at least one agent (blocks 104, 105, 106). Advantageously, the revised dose can be determined as a function of the first input cycle dose(s) and second input determination of the relevancy of the at least one non-pharmacologic modality.

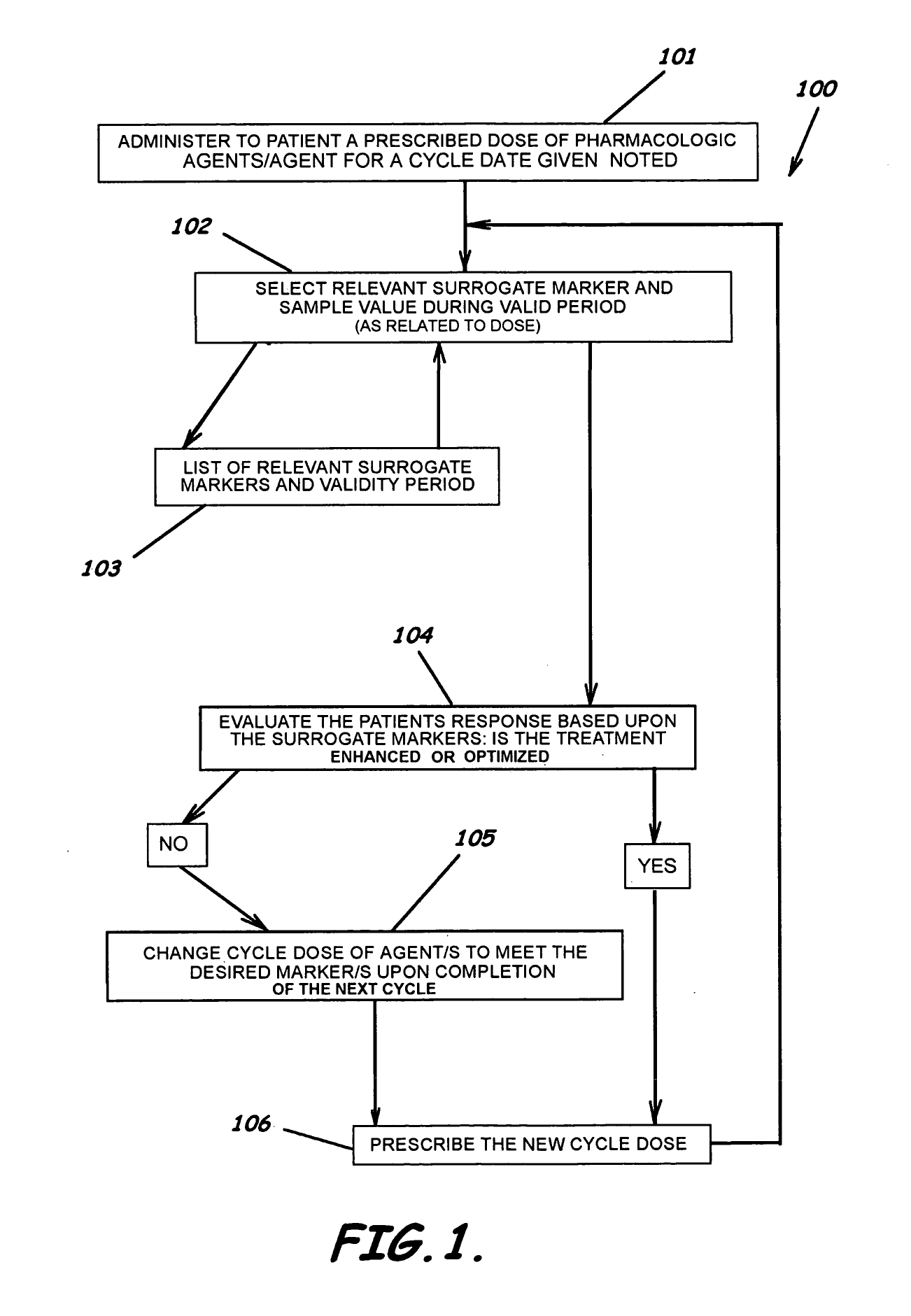

FIG. 1, for example, shows a flow chart 100 of an embodiment of an overall process or method 100 of treating a patient receiving a single or multi-agent therapy to enhance or optimize pharmacologic outcome to reduce the risk of an adverse drug event while assessing the clinical relevancy and validity of the measured at least one surrogate marker. As shown in FIG. 1, a patient receiving a therapy is given a cycle dose of at least one agent (block 101). A relevant surrogate marker is selected and sample value during valid period as related to the dose (block 102). A list of the relevant surrogate markers and validity period (block 103) are checked or selected. At the end of a pre-selected period of time, the patient is examined or evaluated based on at least one surrogate marker (block 104). A determination is made whether the current treatment is significantly enhanced or optimized. If yes, the same cycle dose will remain for the next treatment cycle (block 106). If not, a revised cycle dose will be determined responsive to the effect of the at least one agent and further prescribed to the patient for the next treatment cycle (block 105). In this method 100, for example, a cycle dose is used rather than a current dose. Cycle dose advantageously provides more accurate and effective measurement of the efficacy of the at least one agent during the therapy.

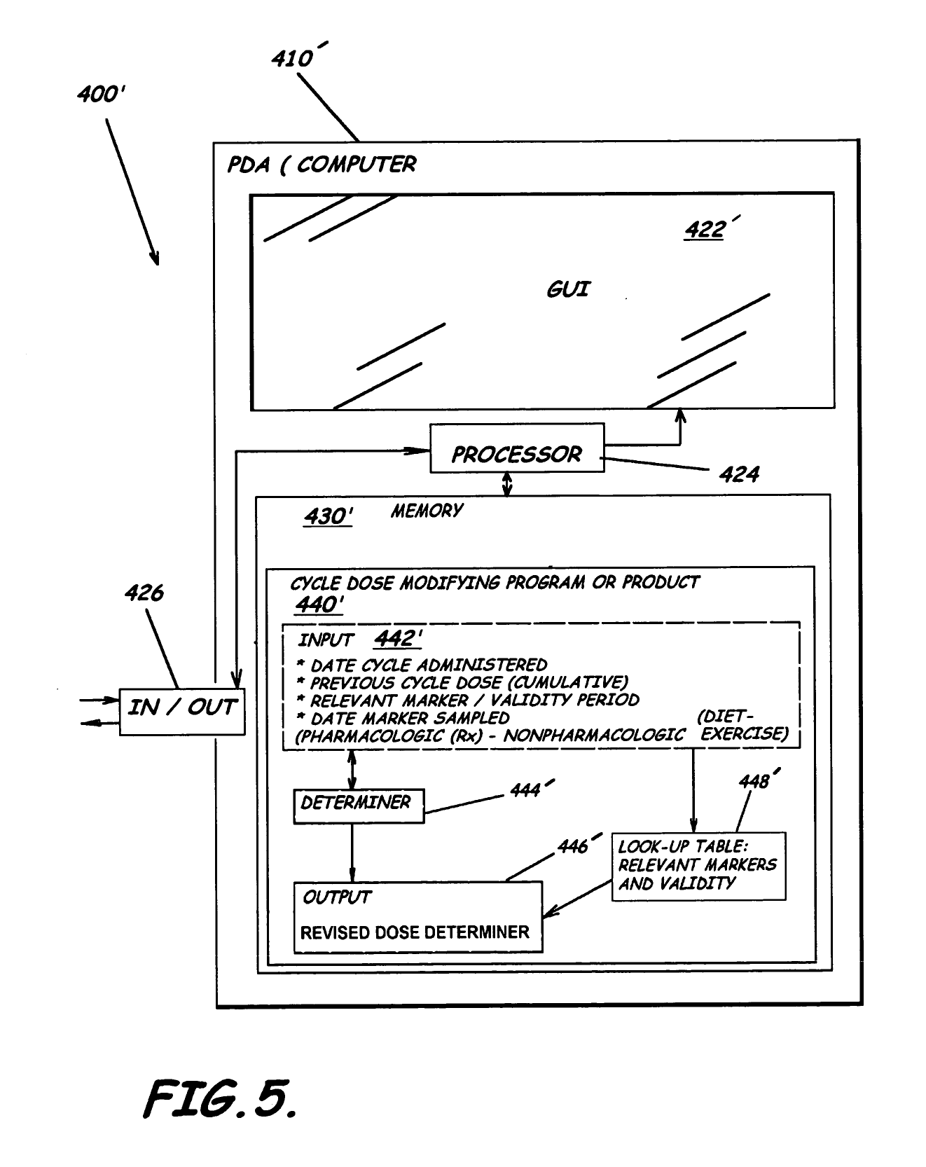

As shown in FIGS. 1-5, an embodiment of the present invention advantageously provides a method 100, 200 for providing a revised dose of at least one agent in a therapy for a patient receiving such a therapy. This method advantageously includes accepting as a first input the patient's cycle dose(s) of the at least one agent (block 101), accepting as a second input at least one non-pharmacologic modality received by the patient (block 201), accepting as a third input determination of the relevancy of at least one surrogate marker indicating a pharmacologic response of the patient (blocks 202, 203), accepting as a fourth input determination of the relevancy of the at least one non-pharmacologic modality indicating achievement of a benefit (blocks 202, 203) and determining a revised cycle dose of the at least one agent (blocks 204, 205, 206). Advantageously, the revised dose can be determined as a function of the cycle dose(s), the at least one non-pharmacologic modality, determination of the relevancy of the at least one surrogate markers and determination of the relevancy of the at least one non-pharmacologic modality. For example, such calculation of the revised dose can be based on the cumulative effect of the at least one agent and the at least one non-pharmacologic modality to be achieved by the single or multi-agent therapy and non-pharmacologic intervention.

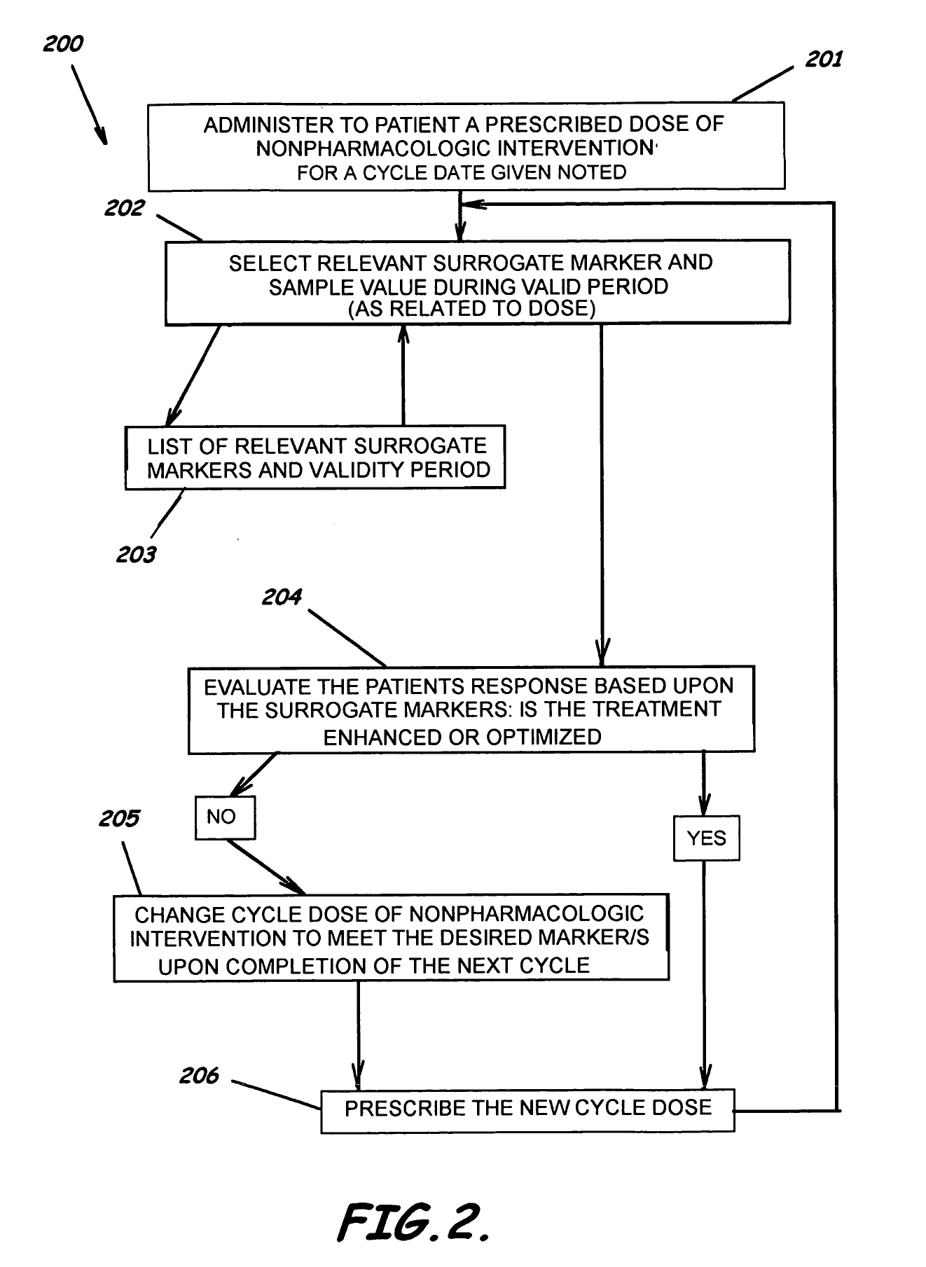

FIG. 2 shows a flow chart of an embodiment of an overall process or method 100, 200 of treating a patient receiving a single or multi-agent therapy to optimize pharmacologic outcome to reduce the risk of an adverse drug event while assessing the clinical relevancy and validity of the measured at least one surrogate marker and of at least one non-pharmacologic modality the patient is receiving. As shown in FIG. 2, a patient receiving a therapy is given a cycle dose of at least one agent (block 201). During the treatment, the patient also receives at least one non-pharmacologic modality. At the end of a pre-selected period of time, the patient is examined based on at least one surrogate marker indicative of the effect of the at least one agent and based on at least one non-phamacologic modality (block 204). A determination is made (block 204) whether the current treatment is optimized. If yes, the same cycle dose will remain for the next treatment cycle (block 206). If not, a revised cycle dose will be determined responsive to the cumulative effect of the at least one agent and at least one non-pharmacologic modality (block 205) and further prescribed to the patient for the next treatment cycle (block 202). In this method 100, 200, for example, a cycle dose is used rather than a current dose. Cycle dose advantageously provides more accurate and effective measurement of the efficacy of the at least one agent during the therapy.

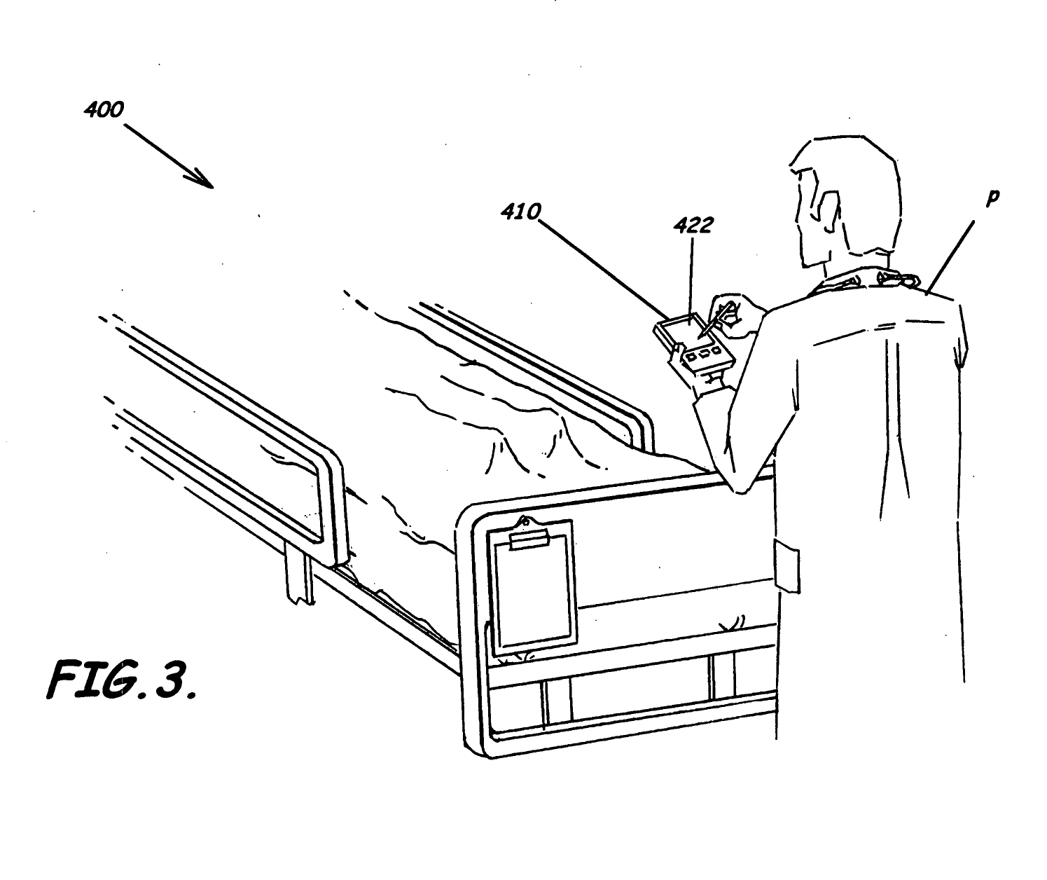

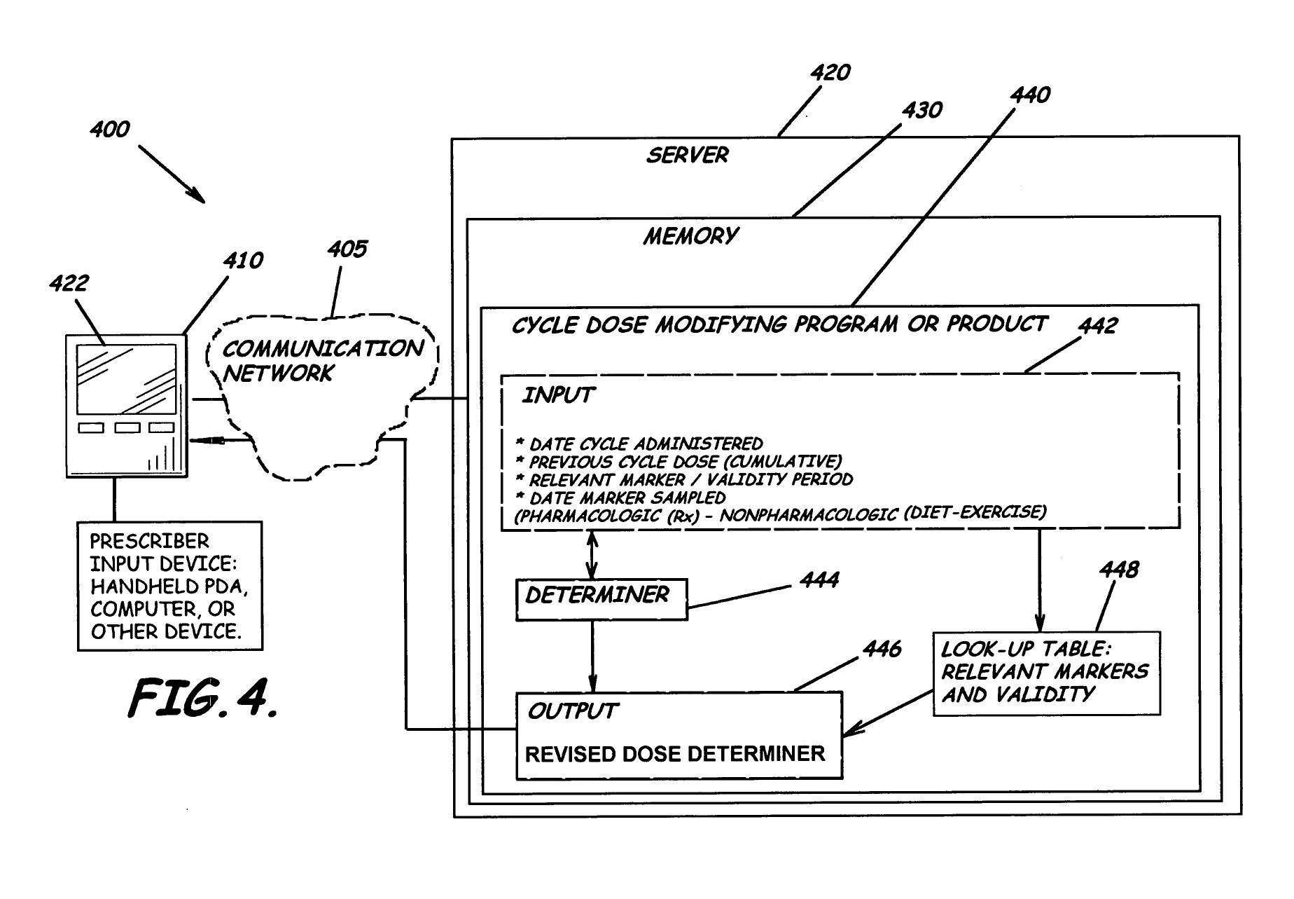

Another embodiment of the present invention advantageously provides a system 400 (see FIGS. 3-4) for revising a cycle dose of at least one agent in a therapy for a patient receiving such a therapy. This system 400 advantageously includes means for accepting as a first input the patient's cycle dose(s) of the at least one agent 442, means for accepting as a second input determination of the relevancy of at least one surrogate marker indicating a pharmacologic response of the patient 442 and means responsive to the first input and second input for calculating a revised cycle dose of the at least one agent, e.g. a calculator such as a determiner 44 and a revised dose determiner 446.

Another embodiment of the present invention advantageously provides a system 400 for revising a cycle dose of at least one agent in a therapy for a patient receiving such a therapy. This system advantageously includes means for accepting as a first input the patient's cycle dose(s) of the at least one agent, means for accepting as a second input at least one non-pharmacologic modality received by the patient, means for accepting as a third input determination of the relevancy of at least one surrogate marker indicating a pharmacologic response of the patient, means for accepting as a fourth input determination of the relevancy of the at least one non-pharmacologic modality indicating achievement of benefit and means for determining a revised cycle dose of the at least one agent as a function of the first input cycle dose(s), the at least one non-pharmacologic modality, determination of the relevancy of the at least one surrogate markers and determination of the relevancy of the at least one non-pharmacologic modality.

Still another embodiment of the present invention advantageously provides a software program 442 for revising a cycle dose of at least one agent in a therapy for a patient receiving such a therapy. This software program 442 advantageously includes means for accepting as a first input the patient's cycle dose(s) of the at least one agent, means for accepting as a second input determination of the relevancy of at least one surrogate marker indicating a pharmacologic response of the patient, and means responsive to the first input and second input for calculating a revised cycle dose of the at least one agent.

Still another embodiment of the present invention advantageously provides a software program 442 for revising a cycle dose of at least one agent in a therapy for a patient receiving such a therapy. This software program 442 advantageously includes means for accepting as a first input the patient's cycle dose(s) of the at least one agent, means for accepting as a second input at least one non-pharmacologic modality received by the patient, means for accepting as a third input determination of the relevancy of at least one surrogate marker indicating a pharmacologic response of the patient, means for accepting as a fourth input determination of the relevancy of the at least one non-pharmacologic modality indicating achievement of benefit and means for determining a revised cycle dose of the at least one agent as a function of the first input cycle dose(s), the at least one non-pharmacologic modality, determination of the relevancy of the at least one surrogate markers and determination of the relevancy of the at least one non-pharmacologic modality.

Yet another embodiment of the present invention advantageously provides a software program for revising a cycle dose of at least one agent in a therapy for a patient receiving such a therapy. This software program advantageously includes a first receiver to accept as a first input the patient's cycle dose(s) of the at least one agent, a second receiver to accept as a second input determination of the relevancy of at least one surrogate marker indicating a pharmacologic response of the patient and a calculator to calculate a revised cycle dose of the at least one agent.

Yet another embodiment of the present invention advantageously provides a software program for revising a cycle dose of at least one agent in a therapy for a patient receiving such a therapy. This software program advantageously includes a first receiver to accept as a first input the patient's cycle dose(s) of the at least one agent, a second receiver to accept as a second input at least one non-pharmacologic modality received by the patient, a third receiver to accept as a third input determination of the relevancy of at least one surrogate marker indicating a pharmacologic response of the patient, a fourth receiver to accept as a fourth input determination of the relevancy of the at least one non-pharmacologic modality indicating achievement of benefit and a calculator to calculate a revised cycle dose of the at least one agent as a function of the first input cycle dose(s), the at least one non-pharmacologic modality, determination of the relevancy of the at least one surrogate markers and determination of the relevancy of the at least one non-pharmacologic modality.

FIG. 3 illustrates a process of receiving data input including cycle dose of at least one agent from a patient. For example, a physician P, health practitioner, or other medical personnel receives a cycle dose input through a handheld device 410. The input means can also be advantageously provided by a laptop or other device as shown in FIG. 4. FIG. 4 illustrates an embodiment of a system 400 for use in calculating revised cycle dose of at least one agent for a patient receiving a therapy according to an embodiment of the present invention. This system 400, for example, advantageously includes input means 410, 410′ for accepting patient's cycle dose, determination of the relevancy of surrogate markers and/or determination of relevancy of non-pharmacologic modalities. The input means 410 of a system 400, 400′ can be provided by a handheld, laptop or other device, e.g., preferably having a display 422, 422′ and/or a graphical user interface (GUI), and/or, in alternative embodiments, can include an input interface 442, 442′ or input/output. Further advantageously, the system also includes determining means which is advantageously provided by a cycle dose modifying software 440. The software 440 is stored in memory 430, 431′, which is contained in a computer defining a server 420 as understood by those skilled in the art or on a handheld doser 410′, or in communication through a network 405 with the server 420, can interface with a processor 424 and a look-up table 448, 448′ for looking up relevant markers and validity. The program product or system 400, 400′, for example, can receive input data from a physician or other person or other device regarding the patient's cycle dose(s) of the at least one agent, at least one non-pharmacologic modality received by the patient, determination of the relevancy of at least one surrogate markers indicating a pharmacologic response of the patient and determination of the relevancy of the at least one non-pharmacologic modality indicating achievement of maximum benefit. Upon receiving the data, the system 400, 400′ further determines, e.g., calculates, a revised dose of the at least one agent based on cumulative effect of the at least one agent and the at least one non-pharmacologic modality.

The following examples are given for the purpose of illustrating various embodiments of the invention and are not meant to limit the present invention in any fashion.

### EXAMPLE 1

Single-Agent Cycle Dose Modifier

Warfarin Sodium (coumadin) is used in this example as a single agent. Table 1 illustrates a method of revising a drug dose in treating a patient with one pharamcologic and non-pharmacological modality to optimize pharmacologic outcome and to reduce the risk of an adverse drug event while assessing the clinical relevancy and validity of the measured surrogate marker.

TABLE 1
SINGLE AGENT CYCLE DOSE MODIFIER

| | Current Cycle | Comparison Cycle | | |
|---|---|---|---|---|
| SINGLE AGENT | Warfarin Sodium | Warfarin Sodium | | |
| Previous Cycle Total Dose Warfarin Sodium/_{PCTD} | 60 | 60 | | dblPreviousDose |
| Current Cycle Total Dose Warfarin Sodium/_{CCTD} | 57 | 57 | | dblCurrertDose |
| Date of Current Cycle Dose | Oct. 21, 2004 | Oct. 21, 2004 | | varDoseDate |
| Cycle Dosing Range Maximum | 160 | 160 | | |
| Multiple of Dose Range Maximum | 1 | 1 | | |
| Cycle Dosing Range Maximum Warfarin Sodium/_{CDRM} | 160 | 160 | | dblRange |
| Inverse Marker | No | No | | bollsInverseMarker |
| Previous Level of Efficacy/_{PLTE} | 4.50 | 4.50 | | dblPreviousLevel |
| Current Level of Efficacy/_{CLTE} | 3.80 | 3.80 | | dblCurrentLevel |
| Date Marker Taken | Oct. 26, 2004 | Oct. 26, 2004 | | varMarkerDate |
| Desired Level of Efficacy/_{DLTE} | 3.00 | 3.00 | | dblDesiredLevel |
| Marker Validity | valid | valid | | compare 2 dayMarkL_Limit & |
| | | | | dayMarkU_Limit |
| New Warfarin Sodium Dose/_{NAD} | 48 | 48 | | New Dose |
| Projected Marker Agent/_{PMA} | 3.00 | 3.00 | | Projected Marker |
| Adjusted Value Warfarin Sodium/_{AVA} | 1.07 | 1.07 | < | Adjusted Value |
| Modified New Dose Warfarin Sodium/_{MND} | 49 | 49 | | Modified New Dose |
| Efficacy/Toxicity Determinant Factor/_{ETD} | 1.00 | 1.00 | | dblEfficacyDeterminantFactor |
| Expected Current Level/ECL | 4.19 | 4.19 | | Expected Current Level |

In Table 1, Previous Cycle Total Dose (PCTD) is a value directly entered without new calculation. It is the cumulative amount of the agent administered during the previous treatment cycle as defined by the total amount of drug given over a fixed period of time, such as in chemotherapy, the total amount, e.g., mg/m², administered throughout the defined regimen period. In this single agent therapy example for chronic disease management, that is, prevention of venous thrombosis with warfarin sodium, the cycle becomes the total amount of warfarin sodium, in milligrams, administered over a seven-day period. The period is determined when the modifier is constructed.

Current Cycle Total Dose (CCTD) is also a value directly entered without new calculation. It is the proposed amount of the drug to be given in the current treatment cycle.

Date of Current Cycle Dose is based on the initiation of the cycle (in this agent/s instance; the cycle was predetermined to be a seven day period).

Cycle Dosing Range Maximum is a value determined when the dose modifier is constructed. It is the maximum dosing range that the patient can receive during that cycle calculated by multiplying the maximum dosing range determined during approval by the number of doses given in each cycle.

Multiple of Dose Range Maximum is the number of cycles received by the patient.

Cycle Dosing Range Maximum (CDRM) is the value determined by multiplying the maximum dosing range per cycle times the number of cycles the patient has received.

Inverse Marker is the indication of a direct or inverse relationship between the dose of the agent and the response of the marker. If there is a direct relationship between dose and marker then NO would be typed in the cell. If there is an inverse relationship between dose and marker then YES would be typed in the cell.

Previous Level of Toxicity-Efficacy (PLTE) is a value directly entered such as from a lab report. It is the numerical value of a marker of toxicity or efficacy reported at the previous dose cycle adjustment (evaluated at a specified elapsed time sufficient to capture the nadir of the dose effect). The surrogate marker is selected when the dose modifier is constructed.

Current Level of Toxicity-Efficacy (CLTE) is also a value directly entered such as from a lab report. It is the numerical value of the marker of toxicity or efficacy reported with the current dose cycle. The surrogate marker is that value that illustrates the expression of the toxicity or efficacy of the agent on a specific and pre-selected value determined when the dose modifier is constructed. The value is selected based upon the agent, its pharmacologic properties and the disease condition under treatment. The confidence or validity of the marker is based upon its maturity of effect in that a nadir or cycle of effect is dynamic and may vary based upon the time span between receipt of the agent and sampling of the value. Therefore the value may be valid or invalid based upon this timing factor. If invalid, then the dose adjustment cannot be made. If valid, then the value is fully appreciated and the dose modification can be calculated. The surrogate marker is selected when the dose modifier is constructed.

Date Marker Taken is the date that the surrogate marker has been measured.

Desired Level of Toxicity-Efficacy (DLTE) is a value directly entered such as by a prescriber. It is the desired or goal value of the marker of toxicity or efficacy that is to be achieved by the dose modification.

Marker Validity is a response recorded in the cell as valid or invalid.

| | | | | | Neutrophil | | Hemoglobin | | | |
|---|---|---|---|---|---|---|---|---|---|---|
| | Max | Marker: | Platelets | | Count (ANC) | | (Hgb) | | INR | |
| Agent | Dose | Inverse | LL | UL | LL | UL | LL | UL | LL | UL |
| Carboplatin | 500 | yes | 17 | 25 | 17 | 25 | 14 | 21 | | |
| Cisplatin | 220 | yes | 18 | 23 | 18 | 23 | 18 | 23 | | |
| Gemzar | 3300 | yes | 7 | 10 | 7 | 10 | 7 | 10 | | |
| Taxol | 500 | yes | 8 | 11 | 8 | 11 | 8 | 11 | | |
| Taxotere | 500 | yes | 6 | 8 | 6 | 8 | 8 | 11 | | |
| Warfarin Sodium | 160 | no | | | | | | | 4 | 5 |

A lookup table has been constructed (see above example), in the form of a table, to represent the relationship of each trial Agent and its qualified Clinical Markers. This table includes information regarding the Maximum Dose and the Inverse Marker Applicability for each Agent. The gray cells on the Cycle Dose Modifier indicate these values.

The table also includes information regarding the nadir of applicability for each Agent—Marker combination. The range, in days and indicated as the Lower Limit and Upper limit, has been determined by calculating a percentage (20%) above and below the true nadir value when only a single value is known. Otherwise, the upper and lower nadir values are used. The relationship of Agents to Markers can best be exhibited by the following Entity Relationship Diagram, to-wit: 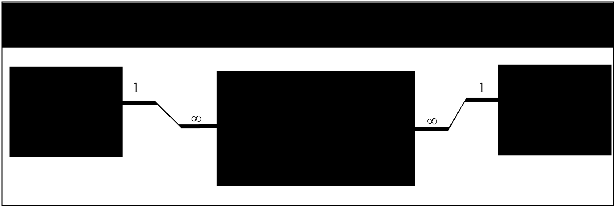
Please note that the relationship of Agents to Associations is one-to-many as is the relationship of Markers to Associations. The values of Upper and Lower limits is shown as a property of the Association between Markers and Agents. The Relevancy of a Marker can be computed using the lookup table by comparing the number of days elapsed between the date of the application of the current dose and the date on which the Marker was taken to the upper and lower values for that Agent Marker Association. If the elapsed days are greater than or equal to the lower limit and less than or equal to the upper limit, then the calculations provided by the Cycle Dose Modifier are said to be relevant and thereby applicable as indicated in the yellow Marker Validity cell. The Relevancy of Cycle Dose Modifier Calculations for Agent 1 is based on the Validity of Marker for Agent 1 calculated by:
=IF(and((($B13−B6)>=VLOOKUP(B$3,Validity!$A$3:$K$8,10)),((B$13−B6)<=VLOOKUP(B$3,Validity!$A$3:$K$8,11))),“valid”,“invalid”)

New Agent Dose (NAD) is calculated by a dose modifier. The new cycle dose of the drug is based upon the plane identity of the agent as well as the observed relationship between dose and marker and determining the relationship as inverse or direct, which equals to,
=IF(B10=“yes”, $B5+((B12−B14)/B12)*B20/(1+($B5/$B9))*$B5,$B5−((B12−B14)/B12)*B20/(1+($B5/$B9))*$B5)
wherein B stands for col. B in Table 1. That is, the new agent dose is calculated by
=IF(InverseMarker=“yes”, CCTD+((CLTE−DLTE)/CLTE)*ETD/(1+(CCTD/CDRM))*CCTD,CCTD−((CLTE−DLTE)/CLTE)*ETD/(1+(CCTD/CDRM))*CCTD)
using the respective value in col. B.

Adjusted Value Agent (AVA) is also calculated by dose modifier. It is the stochastic open loop designed to adjust the dose based upon the individual's actual response versus the expected response, which may increase the dose, reduce the dose or leave the dose the same. The amount of the dose that is reserved is the Variable Sensitivity Value (VSV), determined by the pharmacologic properties of the agent when the dose modifier is built. The adjusted value equals to
=(IF(OR(and((B12<B14),and((IF(B11=0,0,IF($B4=0,0,IF(B10=“Yes”,(−1*(((($B5−$B4)/$B4)*(1+($B4/$B9))*B11))+B11),(((($B5−$B4)/$B4)*(1+($B4/$B9))*B11)+B11)))))>B12))),((20/100)*$B5)*(((IF(B11=0,0, IF($B4=0,0,IF(B10=“Yes”,(−1*(((($B5−$B4)/$B4)*(1+($B4/$B9))*B11))+B11),(((($B5−$B4)/$B4)*(1+($B4/$B9))*B11)+B11)))))−B12)/B12),IF(B12<B14,(−20/100*$B5)*((B12−(IF(B11=0,0,IF($B4=0,0,IF(B10=“Yes”,(−1*(((($B5−$B4)/$B4)*(1+($B4/$B9))*B11))+B11),(((($B5−$B4)/$4)*(1+($B4/$B9))*B11)+B11)))))−B12)/B12)))))/(1.3\hat{ }($B9))
wherein B stands for col. B in Table 1. That is, the adjusted value is calculated by
=(IF(OR(and((CLTE<DLTE),and((IF(PLTE=0,0,IF(PCTD=0,0,IF(InverseMarker=“Yes”,(−1*((((CCTD−PCTD)/PCTD)*(1+(PCTD/CDRM))*PLTE))+PLTE),((((CCTD−PCTD)/PCTD)*(1+(PCTD/CDRM))*PLTE)+PLTE)))))<CLTE))),((20/100)*CCTD)*(((IF(PLTE=0,0, IF(PCTD=0,0,IF(InverseMarker=“Yes”,(−1*((((CCTD−PCTD)/PCTD)*(1+(PCTD/CDRM))*PLTE))+PLTE),((((CCTD−PCTD)/PCTD)*(1+(PCTD/CDRM))*PLTE)+PLTE)))))−CLTE)/CLTE),IF(CLTE<DLTE,(−20/100*CCTD)*((CLTE−(IF(PLTE=0,0,IF(PCTD=0,0,IF(InverseMarker=“Yes”,(−1*((((CCTD−PCTD)/PCTD)*(1+(PCTD/CDRM))*PLTE))+PLTE),((((CCTD−PCTD)/PCTD)*(1+(PCTD/CDRM))*PLTE)+PLTE))))))/CLTE),IF((IF(PLTE=0,0,IF(PCTD=0,0,IF(InverseMarker=“Yes”,(−1*((((CCTD−PCTD)/PCTD)*1+(PCTD/CDRM))*PLTE))+PLTE),((((CCTD−PCTD)/PCTD)*(1+(PCTD/CDRM))*PLTE)+PLTE)))))>,(−(20/100)*CCTD)*((CLTE−(IF(PLTE=0,0,IF(PCTD=0,0,IF(InverseMarker=“Yes”,(−1*((((CCTD−PCTD)/PCTD)*(1+(PCTD/CDRM))*PLTE))+PLTE),((((CCTD−PCTD)/PCTD)*(1+(PCTD/CDRM))*PLTE)+PLTE))))))/CLTE),((20/100)*CCTD)*(((IF(PLTE=0,0,IF(PCTD=0,0,IF(InverseMarker=“Yes”,(−1*((((CCTD−PCTD)/PCTD)*(1+(PCTD/CDRM))*PLTE))+PLTE),((((CCTD−PCTD)/PCTD)*(1+(PCTD/CDRM))*PLTE)+PLTE)))))−CLTE)/CLTE)))))/(1.3\hat{ }(CCTD/CDRM))
using the respective value in Col. B.

Modified New Dose (MND) is also calculated by a dose modifier. The newly calculated drug dose is based upon the fit of the drug on its dose response curve, the individualization required for this patient to achieve the desired marker as well as the observed relationship between dose and marker thus determining the relationship as inverse or direct, which equals to
=IF(B10=“Yes”,B16−B18,B16+B18)
that is,
=IF(InverseMarker=“Yes”,NAD−AVA, NAD+AVA)

Efficacy Toxicity Determinant (ETD) is a value determined by the prescriber. Set at an initial value of 1, ETD is then adjusted based upon actual case scenarios with a full data set to adjust the value to identify the contribution to the toxicity or efficacy until the actual value is achieved. It can be used to modify dose based upon non-pharmacologic effects such as diet, exercise and activity as determined by the prescriber.

Expected Current Level (ECL) is a comparative operator used to illustrate the mathematical expectation of the patient's response to the actual patient response to classify their individual response characteristics. Which equals,
=IF(C11=0,0, IF(C4=0,0,IF(C10=“Yes”,(−1*((((C5−C4)/C4)*(1+(C4/C9))*C11))+C11),((((C5−C4)/C4)*(1+(C4/C9))*C11)+C11))))
That is,
=IF(PLTE=0,0,IF(PCTD=0,0,IF(InverseMarker=“Yes”,(−1*((((CCTD−PCTD)/PCTD)*(1+(PCTD/CDRM))*PLTE))+PLTE),((((CCTD−PCTD)/PCTD)*(1+(PCTD/CDRM))*PLTE)+PLTE))))

### EXAMPLE 2

Dual-Agent Cycle Dose Modifier

Gemzar and Taxol are used in this example as dual agents. Table 2 illustrates a method of revising a drug dose in treating a patient with two pharamcologic and non-pharmacological modalities to optimize pharmacologic outcome, e.g., to reduce risk of an adverse drug event while assessing the clinical relevancy and validity of the measured surrogate markers.

TABLE 2
Chemotherapeutic Multi-Agent Cycle Dose Modifier

| NEW DOUBLET | | Taxol | Gemzar | |
|---|---|---|---|---|
| Previous Cycle Total Dose/PCTDA1, 2 | | 385 | 7650 | dblPreviousDose A1, A2 |
| Current Cycle Total Dose/CCTDA1, 2 | | 300 | 6200 | dblCurrentDose A1, A2 |
| Date of Current Cycle Dose | | Oct. 21, 2004 | Oct. 21, 2004 | varDoseDate A1, A2 |
| Cycle Dosing Range Maximum | | 500 | 3300 | dblMaxRange A1, A2 |
| Multiple of Dose Range Maximum | | 1 | 3 | intRangeModifier A1, A2 |
| Adj Cycle Dosing Range Maximum/CDRMA1, 2 | | 500 | 9900 | dblRange A1, A2 |
| ANC | Inverse MarkerM1 | Yes | | bollsInverseMarker M1 |
| | Previous Level of Toxicity/_{PLTEM1} | 1.00 | | dblPreviousLevel M1 |
| | Current Level of Toxicity/_{CLTEM1} | 1.50 | | dblCurrentLevel M1 |
| | Date Marker Taken | Oct. 30, 2004 | | varMarkerDate M1 |
| | Desired Level of Toxicity/_{DLTEM1} | 2.00 | | dblDesiredLevel M1 |
| | Agent Fraction/FA1, 2 | 0.49 | 0.51 | |
| | Total Share of Effect/TSEA1, 2 | 0.24 | 0.26 | |
| | Proportion of Effect/PEA1, 2 | 0.48 | 0.52 | |
| | Marker Validity | valid | valid | compare 2 dayMarkL_Limit |
| | | | | & dayMarkU_Limit |
| | New Dose/NAD1, 2 | 270 | 5703 | |
| | Projected Marker/PMA1, 2 | 1.74 | 1.70 | |
| | Adjusted Value/AVA1, 2 | −4 | −115 | |
| | Modified New Dose/MNDA1, 2 | 274 | 5818 | |

In Table 2, Previous Cycle Total Dose Agent 1 (PCTD 1) is a value of Taxol directly entered without a new calculation. It is the cumulative amount of Taxol administered during the previous treatment cycle as defined by the total amount of drug given over a fixed period of time, the total amount, e.g., mg/m², administered throughout the defined regimen cycle period. The cycle is defined when the dose modifier is constructed.

Previous Cycle Total Dose Agent 2 (PCTD2) is a value of Gemzar directly entered without a new calculation. It is the cumulative amount of Gemzar administered during the previous treatment cycle as defined by the total amount of drug given over a fixed period of time, the total amount, e.g., mg/m², administered throughout the defined regimen cycle period. The cycle is defined when the dose modifier is constructed.

Current Cycle Total Dose Agent 1 (CCTD1) is a value of Taxol directly entered without a new calculation. It is the proposed amount of Taxol to be given in the current treatment cycle.

Current Cycle Total Dose Agent 2 (CCTD2) is a value of Gemzar directly entered without a new calculation. It is the proposed amount of Gemzar to be given in the current treatment cycle.

Date of Current Cycle Dose is based on the initiation of the cycle (in this instance; the cycle was predetermined to be a fourteen day period).

Previous Level of Toxicity-Efficacy (PLTE) is a value directly entered such as from a lab report. It is the numerical value of the marker of toxicity or efficacy reported at the previous dose cycle adjustment (evaluated at a specified elapsed time sufficient to capture the nadir of the dose effect). The surrogate marker is selected when the dose modifier is constructed.

Current Level of Toxicity-Efficacy (CLTE) is a value directly entered such as from a lab report. It is the numerical value of the marker of toxicity or efficacy reported with the current dose cycle. The surrogate marker is that value that illustrates the expression of the toxicity or efficacy of the agent on a specific and pre-selected value determined when the dose modifier is constructed. The value is selected based upon the agent, its pharmacologic properties and the disease condition under treatment. The confidence or validity of the marker is based upon its maturity of effect in that a nadir or cycle of effect is dynamic and may vary based upon the time span between receipt of the agent and sampling of the value. Therefore, the value may be valid or invalid based upon this timing factor. If invalid, then the dose adjustment cannot be made. If valid, then the value is fully appreciated and the dose modification can be calculated. The surrogate marker is selected when the dose modifier is constructed.

Date Marker Taken is the date that the surrogate marker has been measured.

Desired Level of Toxicity-Efficacy (DLTE) is a value directly entered by the prescriber. It is the desired or goal value of the marker of toxicity or efficacy that is to be achieved by the dose modification.

Cycle Dosing Range Maximum Agent 1 (CDRM1) is a value of Taxol determined when the dose modifier is constructed. It is the maximum dosing range of the first agent (i.e., Taxol) the patient can receive during the cycle calculated by multiplying the maximum dosing range determined during approval by the number of doses given in each cycle.

Cycle Dosing Range Maximum Agent 2 (CDRM2) is a value of Gemzar determined when the dose modifier is constructed. It is the maximum dosing range of the second agent (i.e., Gemzar) the patient can receive during the cycle calculated by multiplying the maximum dosing range determined during approval by the number of doses given in each cycle.

Fraction of the Dose Agent 1 (FA1) is a value of Taxol calculated by a dose modifier. The Fraction of the Dose Agent 1 (i.e., Taxol) is used to determine the amount of the individual drugs proportion within the combined dose. This number is calculated by dividing the current cycle total dose of the agent by the cycle dose maximum range of the agent divided by the sum of the current cycle total dose of the agent by cycle dose maximum range of the agent and the current cycle total dose of the second agent by cycle dose maximum range of the second agent. Which equals to,
=($E4/$E8)/($E4/$E8+$F4/$F8)
That is,
=(CCTDA1/CDRMA1)/(CCTDA1/CDRMA1+CCTDA2/CDRMA2)

Fraction of the Dose Agent 2 (FA2) is a value of Gemzar calculated by a dose modifier. The Fraction of the Dose Agent 2 (i.e., Gemzar) is used to determine the amount of the individual drugs proportion within the combined dose. This number is calculated by dividing the current cycle total dose of the agent by the cycle dose maximum range of the agent divided by the sum of the current cycle total dose of the agent by cycle dose maximum range of the agent and the current cycle total dose of the second agent by cycle dose maximum range of the second agent. Which equals,
=($F4/$F8)/($E4/$E8+$F4/$F8)
That is,
=(CCTDA2/CDRMA2)/(CCTDA1/CDRMA1+CCTDA2/CDRMA2)

Total Share of Effect Agent 1 (Taxol) (TSE1) is calculated by a dose modifier. This fraction value is used to describe the percentage of effect attributed to the individual drug within the mix based upon previous calculation using the fraction and the fit to actual experience with the drug mix and marker and the relationship of the drugs effect to the marker. This calculation is reflected on the data sheet and is part of “building” each dose modifier. The determinant of efficacy/toxicity is combination specific and unique to the individual marker.

Initially based upon the assessment of the agents' individual characteristics, peer reviewed publications and available clinical experience can identify the likelihood and degree of positive and negative effects (markers) of all agents in the combination therapy. With this approximation, TSE1 value is applied to an existing dataset and correlation, standard deviation and standard error are calculated. Then the number is adjusted to derive the optimal determinant value.

The process is repeated for each individual marker, as the value is unique to each individual marker when the dose modifier is constructed. Which equals,
=($E4/$E8)/($E4/$E8+$F4/$F8)*E15
That is,
=(CCTDA1/CDRMA1)/(CCTDA1/CDRMA1+CCTDA2/CDRMA2)*FA1

Total Share of Effect Agent 2 (Gemzar) (TSE2) is calculated by a dose modifier. This fraction value is used to describe the percentage of effect attributed to the individual drug within the mix based upon previous calculation using the fraction and the fit to actual experience with the drug mix and marker and the relationship of the drugs effect to the marker. This calculation is reflected on the data sheet and is part of “building” each dose modifier. The determinant of efficacy/toxicity is a combination specific and unique to the individual marker.

Initially based upon the assessment of the agents' individual characteristics, peer reviewed publications and available clinical experience can identify the likelihood and degree of positive and negative effects (markers) of all agents in the combination therapy. With this approximation, TSE2 value is applied to an existing dataset and correlation, standard deviation and standard error are calculated. Then the number is adjusted to derive the optimal determinant value.

The process is repeated for each individual marker, as the value is unique to each individual marker when the dose modifier is constructed. Which equals,
=($F4/$F8)/($E4/$E8+$F4/$F8)*F15
That is,
=(CCTDA2/CDRMA2)/(CCTDA1/CDRMA1+CCTDA2/CDRMA2)*FA2

Proportion of Effect 1 (Agent 1-Taxol) (PE1) is calculated by a dose modifier. Once the total share of effect is determined then each individual agent's share of that effect is divided by the total effect to determine what proportion of the change in the marker is that of the individual agent. PE1 equals to the total share of the affect of the first agent divided by the total share of affect for agent one plus the total share of affect for agent two. Which equals,
=E16/(E16+F16)
That is,
=TSEA1/(TSEA1+TSEA2)

Proportion of Effect 2 (Agent 2-Gemzar) (PE2) is calculated by a dose modifier. Once the total share of effect is determined then each individual agent's share of that effect is divided by the total effect to determine what proportion of the change in the marker is that of the individual agent. PE2 equals to the total share of the affect of the second agent divided by the total share of affect for agent one plus the total share of affect for agent two. Which equals,
=F16/(E16+F16)
That is,
=TSEA2/(TSEA1+TSEA2)

Marker Validity is a response recorded in the cell as valid or invalid.

| | | | | | Neutrophil | | Hemoglobin | | | |
|---|---|---|---|---|---|---|---|---|---|---|
| | Max | Marker: | Platelets | | Count (ANC) | | (Hgb) | | INR | |
| Agent | Dose | Inverse | LL | UL | LL | UL | LL | UL | LL | UL |
| Carboplatin | 500 | yes | 17 | 25 | 17 | 25 | 14 | 21 | | |
| Cisplatin | 220 | yes | 18 | 23 | 18 | 23 | 18 | 23 | | |
| Gemzar | 3300 | yes | 7 | 10 | 7 | 10 | 7 | 10 | | |
| Taxol | 500 | yes | 8 | 11 | 8 | 11 | 8 | 11 | | |
| Taxotere | 500 | yes | 6 | 8 | 6 | 8 | 8 | 11 | | |
| Warfarin Sodium | 160 | no | | | | | | | 4 | 5 |

A lookup table has been constructed (see above example), in the form of a table, to represent the relationship of each trial Agent and its qualified Clinical Markers. This table includes information regarding the Maximum Dose and the Inverse Marker Applicability for each Agent. The gray cells on the Cycle Dose Modifier indicate these values.

The table also includes information regarding the nadir of applicability for each Agent—Marker combination. The range, in days and indicated as the Lower Limit and Upper limit, has been determined by calculating a percentage (20%) above and below the true nadir value when only a single value is known. Otherwise, the upper and lower nadir values are used. The relationship of Agents to Markers can best be exhibited by the following Entity Relationship Diagram, to-wit: 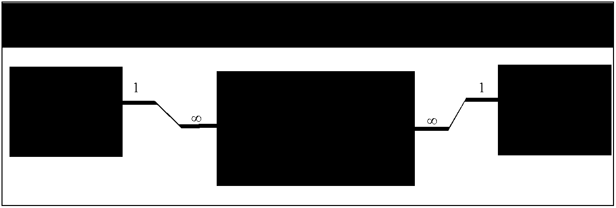
Please note that the relationship of Agents to Associations is one-to-many as is the relationship of Markers to Associations. The values of Upper and Lower limits is shown as a property of the Association between Markers and Agents. The Relevancy of a Marker can be computed using the lookup table by comparing the number of days elapsed between the date of the application of the current dose and the date on which the Marker was taken to the upper and lower values for that Agent Marker Association. If the elapsed days are greater than or equal to the lower limit and less than or equal to the upper limit, then the calculations provided by the Cycle Dose Modifier are said to be relevant and thereby applicable as indicated in the yellow Marker Validity cell. The Relevancy of Cycle Dose Modifier Calculations for Agent 1 is based on the Validity of Marker for Agent 1 calculated by:
=IF(AND((($E13−E5)>=VLOOKUP(E$2,Validity!$A$3:$K$8,6)),((E$13−E5)<=VLOOKUP(E$2,Validity!$A$3:$K$8,7))),“valid”,“invalid”)
The Relevancy of Cycle Dose Modifier Calculations for Agent 2 is based on the Validity of Marker for Agent 2 calculated by:
=IF(AND((($E13−F5)>=VLOOKUP(F$2,Validity!$A$3:$K$8,6)),((F$13−F5)<=VLOOKUP(F$2,Validity!$A$3:$K$8,7))),“valid”,“invalid”)

NAD1 New Agent Dose 1 (Agent 1-Taxol) (NAD1) is calculated by a dose modifier. The new cycle dose of Agent 1 (Taxol) is calculated based upon the plane identity of the agent as well as the observed relationship between dose and marker and determining the relationship as inverse or direct, which equals,
=IF(E$10=“yes”,$E4+((E$12−E$14)/E$12)*E17/(1+($E4/$E8))*$E4,$E4−((E$12−E$14)/E$12)*E17/(1+($E4/$E8))*$E4)
That is,
=IF(INVERSEMARKER=“yes”,CCTDA1+((CLTE−DLTE)/CLTE)*PEA1/(1+(CCTDA1/CDRMA1))*CCTDA1,CCTDA1−((CLTE−DLTE)/CLTE)*PEA1/(1+(CCTDA1/CDRMA1))*CCTDA1)

New Agent Dose 2 (Agent 2-Gemzar) (NAD2) is also calculated by a dose modifier. The new cycle dose of Agent 2 (Gemzar) is calculated based upon the plane identity of the agent as well as the observed relationship between dose and marker and determining the relationship as inverse or direct, which equals,
=IF(E$10=“yes”,$F4+((E$12−E$14)/E$14)*F17/(1+($F4/$F8))*$F4,$F4−((E$12−E$14)/$E8)*F17/(1+($F4/$F8))*$F4)
That is,
=IF(INVERSEMARKER=“yes”,CCTDA2+((CLTE−DLTE)/DLTE)*PEA2/(1+(CCTDA2/CDRMA2))*CCTDA2,CCTDA2−((CLTE−DLTE)/CDRMA1)*PEA2/(1+(CCTDA2/CDRMA2))*CCTDA2)

Projected Marker Agent 1 (Taxol) (PM1) is calculated by a dose modifier. It is the expected marker value for Agent 1 based upon the new dose calculation. Which equals,
=IF(OR(and(E$10=“YES”,E22=(2*$E4))),−1*((((((E22)−$E4)/$E4)*(1+(($E4/$E8))*1))*E$12)−E$12),IF(OR(and(E$10=“YES”,E22=(0.5*$E4))),−1*((((((E22)−$E4)/$E4)*(1+(($E4/$E8))*1))*E$12)−E$12),IF(E$10=“YES”,−1*((((((E22+E21)−$E4)/$E4)*(1+(($E4/$E8))*1))*E$12)−E$12),IF(OR(and(E$10=“NO”,E22=(2*$E4))),((((E22−$E4)/$E4)*(1+(($E4/$E8))*1))*E$12)+E$12,IF(OR(and(E$10=“NO”, E22=(0.5*$E4))),((((E22−$E4)/$E4)*(1+(($E4/$E8))*1))*E$12)+E$12,(((((E22−E21)−$E4)/$E4)*(1+(($E4/$E8))*1))*E$12)+E$12)))))
That is,
=IF(OR(and(INVERSEMARKER=“YES”,MNDA1=(2*CCTDA1))),−1*((((((MNDA1)−CCTDA1)/CCTDA1)*(1+((CCTDA1/CDRMA1))*1))*CLTE)−CLTE),IF(OR(and(INVERSEMARKER=“YES”,MNDA1=(0.5*CCTDA1))),−1*((((((MNDA1)−CCTDA1)/CCTDA1)*(1+((CCTDA1/CDRMA1))*1))*CLTE)−CLTE),IF(INVERSEMARKER=“YES”,−1*((((((MNDA1+AVA1)−CCTDA

Projected Marker Agent 2 (Gemzar) (PM2) is calculated by a dose modifier. It is the expected marker value for Agent 2 based upon the new dose calculation. Which equals,
=IF(OR(and(E$10=“YES”,F22=(2*$F4))),−1*((((((F22)−$F4)/$F4)*(1+(($F4/$F8))*1))*E$12)−E$12),IF(OR(and(E$10=“YES”,F22=(0.5*$F4))),−1*((((((F22)−$F4)/$F4)*(1+(($F4/$F8))*1))*E$12)−E$12),IF(E$10=“YES”,−1*((((((F22+F21)−$F4)/$F4)*(1+(($F4/$F8))*1))*E$12)−E$12),IF(OR(and(E$10=“NO”,F22=(2*$F4))),((((F22−$F4)/$F4)*(1+(($F4/$F8))*1))*E$12)+E$12,IF(OR(and(E$10=“NO”,F22=(0.5*$F4))),((((F22−$F4)/$F4)*(1+(($F4/$F8))*1))*E$12)+E$12,(((((F22−F21)−$F4)/$F4)*(1+(($F4/$F8))*1))*E$12)+E$12)))))
That is,
=IF(OR(and(INVERSEMARKER=“YES”,MNDA2=(2*CCTDA2))),−1*((((((MNDA2)−CCTDA2)/CCTDA2)*(1+((CCTDA2/CDRMA2))*1))*CLTE)−CLTE),IF(OR(and(INVERSEMARKER=“YES”,MNDA2=(0.5*CCTDA2))),−1*((((((MNDA2)−CCTDA2)/CCTDA2)*(1+((CCTDA2/CDRMA2))*1))*CLTE)−CLTE), IF(INVERSEMARKER=“YES”,−1*((((((MNDA2+AVA2)−CCTDA2)/CCTDA2)*(1+((CCTDA2/CDRMA2))*1))*CLTE)−CLTE),IF(OR(and(INVERSEMARKER=“NO”,MNDA2=(2*CCTDA2))),((((MNDA2−CCTDA2)/CCTDA2)*(1+((CCTDA2/CDRMA2))*1))*CLTE)+CLTE,IF(OR(and(INVERSEMARKER=“NO”,MNDA2=(0.5*CCTDA2))),((((MNDA2−CCTDA2)/CCTDA2)*(1+((CCTDA2/CDRMA2))*1))*CLTE)+CLTE,(((((MNDA2−AVA2)−CCTDA2)/CCTDA2)*(1+((CCTDA2/CDRMA2))*1))*CLTE)+CLTE)))))

Adjusted Value Agent 1 (Taxol) (AVA1) is calculated by a dose modifier. It is the stochastic open loop designed to adjust the dose of Taxol based upon the individual's actual response versus the expected response, which may increase the dose, reduce the dose or leave the dose the same. The amount of the dose that is reserved is Variable Sensitivity Value (VSV), which is determined by the pharmacologic properties of the agent when the dose modifier is built. Which equals,
=(IF(OR(and((E$12<E$14),and((IF(E$11=0,0,IF($E3=0,0,IF(E$10=“Yes”,(−1*(((($E4−$E3)/$E3)*(1+($E3/$E8))*E$11))+E$11),(((($E4−$E3)/$E3)*(1+($E3/$E8))*E$11)+E$11)))))>E$12))),((20/100)*$E4)*(((IF(E$11=0,0,IF($E3=0,0,IF(E$10=“Yes”,(−1*(((($E4−$E3)/$E3)*(1+($E3/$E8))*E$11))+E$11),(((($E4−$E3)/$E3)*(1+($E3/$E8))*E$11)+E$11)))))−E$12)/E$12),IF(E$12<E$14,(−20/100*$E4)*((E$12−(IF(E$11=0,0,IF($E3=0,0,IF(E$10=“Yes”,(−1*(((($E4−$E3)/$E3)*(1+($E3/$E8))*E$11))+E$11),(((($E4−$E3)/$E3)*(1+($E3/$E8))*E$11)+E$11))))))/E$12),IF((IF(E$11=0,0,IF($E3=0,0,IF(E$10=“Yes”,(−1*(((($E4−$E3)/$E3)*(1+($E3/$E8))*E$11))+E$11),(((($E4−$E3)/$E3)*(1+($E3/$E8))*E$11)+E$11)))))>E$12,(−(20/100)*$E4)*((E$12−(IF(E$11=0,0,IF($E3=0,0,IF(E$10=“Yes”,(−1*(((($E4−$E3)/$E3)*(1+($E3/$E8))*E$11))+E$11),(((($E4−$E3)/$E3)*(1+($E3/$E8))*E$11)+E$11))))))/E$12),((20/100)*$E4)*(((IF(E$11=0,0,IF($E3=0,0,IF(E$10=“Yes”,(−1*(((($E4−$E3)/$E3)*(1+($E3/$E8))*E$11))+E$11),(((($E4−$E3)/$E3)*(1+($E3/$E8))*E$11)+E$11)))))−E$12)/E$12)))))/(1.3\hat{ }($E4/$E8))
That is,
=(IF(OR(and((CLTE<DLTE),and((IF(PLTE=0,0,IF(PCTDA1=0,0,IF(INVERSEMARKER=“Yes”,(−1*((((CCTDA1−PCTDA1)/PCTDA1)*(1+(PCTDA1/CDRMA1))*PLTE))+PLTE),((((CCTDA1−PCTDA1)/PCTDA1)*(1+(PCTDA1/CDRMA1))*PLTE)+PLTE)))))>CLTE))),((20/100)*CCTDA1)*(((IF(PLTE=0,0,IF(PCTDA1=0,0, IF(INVERSEMARKER=“Yes”,(−1*((((CCTDA1−PCTDA1)/PCTDA1)*(1+(PCTDA1/CDRMA1))*PLTE))+PLTE),((((CCTDA1−PCTDA1)/PCTDA1)*(1+(PCTDA1/CDRMA1))*PLTE)+PLTE)))))−CLTE)/CLTE),IF(CLTE<DLTE,(−20/100*CCTDA1)*((CLTE−(IF(PLTE=0,0,IF(PCTDA1=0,0,IF(INVERSEMARKER=“Yes”,(−1*((((CCTDA1−PCTDA1)/PCTDA1)*(1+(PCTDA1/CDRMA1))*PLTE))+PLTE),((((CCTDA1−PCTDA1)/PCTDA1)*(1+(PCTDA1/CDRMA1))*PLTE)+PLTE))))))/CLTE),IF((IF(PLTE=0,0,IF(PCTDA1=0,0,IF(INVERSEMARKER=“Yes”,(−1*((((CCTDA1−PCTDA1)/PCTDA1)*(1+(PCTDA1/CDRMA1))*PLTE))+PLTE),((((CCTDA1−PCTDA1)/PCTDA1)*(1+(PCTDA1/CDRMA1))*PLTE)+PLTE)))))>CLTE,(−(20/100)*CCTDA1)*((CLTE−(IF(PLTE=0,0,IF(PCTDA1=0,0,IF(INVERSEMARKER=“Yes”,(−1*((((CCTDA1−PCTDA1)/PCTDA1)*(1+(PCTDA1/CDRMA1))*PLTE))+PLTE),((((CCTDA1−PCTDA1)/PCTDA1)*(1+(PCTDA1/CDRMA1))*PLTE)+PLTE))))))/CLTE),((20/100)*CCTDA1)*(((IF(PLTE=0,0,IF(PCTDA1=0,0,IF(INVERSEMARKER=“Yes”,(−1*((((CCTDA1−PCTDA1)/PCTDA1)*(1+(PCTDA1/CDRMA1))*PLTE))+PLTE),((((CCTDA1−PCTDA1)/PCTDA1)*(1+(PCTDA1/CDRMA1))*PLTE)+PLTE)))))−CLTE)/CLTE)))))/(1.3\hat{ }(CCTDA1/CDRMA1))

Adjusted Value Agent 2 (Gemzar) (AVA2) is calculated by a dose modifier. It is the stochastic open loop designed to adjust the dose of Gemzar based upon the individual's actual response versus the expected response, which may increase the dose, reduce the dose or leave the dose the same. The amount of the dose that is reserved is Variable Sensitivity Value (VSV), which is determined by the pharmacologic properties of the agent when the dose modifier is built. Which equals,
=(IF(OR(and((E$12<E$14),and((IF(E$11=0,0,IF($F3=0,0,IF(E$10=“Yes”,(−1*(((($F4−$F3)/$F3)*(1+($F3/$F8))*E$11))+E$11),(((($F4−$F3)/$F3)*(1+($F3/$F8))*E$11)+E$11)))))>E$12))),((20/100)*$F4)*(((IF(E$11=0,0,IF($F=$F8))*E$11))+E$11),(((($F4−$F3)/$F3)*(1+($F3/$F8))*E$11)+E$11))))))/E$12),((20/100)*$F4)*(((IF(E$11=0,0,IF($F3=0,0,IF(E$10=“Yes”,(−1*(((($F4−$F3)/$F3)*(1+($F3/$F8))*E$11))+E$11),(((($F4−$F3)/$F3
That is,
=(IF(OR(and((CLTE<DLTE),and((IF(PLTE=0,0,IF(PCTDA2=0,0,IF(INVERSEMARKER=“Yes”,(−1*((((CCTDA2−PCTDA2)/PCTDA2)*(1+(PCTDA2/CDRMA2))*PLTE))+PLTE),((((CCTDA2−PCTDA2)/PCTDA2)*(1+(PCTDA2/CDRMA2))*PLTE)+PLTE)))))>CLTE))),((20/100)*CCTDA2)*(((IF(PLTE=0,0,IF(PCTDA2=0,0,IF(INVERSEMARKER=“Yes”,(−1*((((CCTDA2−PCTDA2)/PCTDA2)*1+(PCTDA2/CDRMA2))*PLTE)+PLTE)))))>CLTE))),((20/100)*CCTDA2)*(((IF(PLTE=0,0,IF(PCTDA2=0,0IF(INVERSEMARKER=“Yes”,(−1*((((CCTDA2−PCTDA2)/PCTDA2)*(1+(PCTDA2/CDRMA2))*PLTE)+PLTE)))))−CLTE)/CLTE),IF(CLTE<DLTE,(−20/100*CCTDA2)*((CLTE−(IF(PLTE=0,0IF(PCTDA2=0,0,IF(INVERSEMARKER=“Yes”,(−1*((((CCTDA2−PCTDA2)/PCTDA2)*(1+(PCTDA2/CDRMA2))*PLTE))+PLTE),((((CCTDA2−PCTDA2)/PCTDA2)*(1+(PCTDA2/CDRMA2))*PLTE)+PLTE))))))/CLTE),IF((IF(PLTE=0,0IF(PCT DA2=0,0IF(INVERSEMARKER)=“Yes”,(−1*((((CCTDA2−CTDA2)/PDCTDA2)*(1+(PCTDA2/CDRMA2))*PLTE))+PLTE),((((CCTDA2−PCTDA2)/PCTDA2)*(1+(PCTDA2/CDRMA2))*PLTE)+PLTE)))))>CLTE,(−(20/100)*CCTDA2)*((CLTE−(if(PLTE=0,0,IF(PCTDA2=0,0(IF(INVERSEMARKER=“Yes”,(−1*((((CCTDA2−PCTDA2)/PCTDA2)*(1+(pctda2/CDRMA2))*PLTE))+PLTE),((((CCTDA2=PCTDA2)/PCTDA2)*(1+(PCTDA2/CDRMA2))*PLTE)+PLTE)))))/CLTE),((20/100)*CCTDA2)*(((IF (PLTE=0,0,IF(PCTDA2=0,0IF(INVERSEMARKER=“Yes”,(−1*((((CCTDA2−PCTDA2)/PCTDA2)*(1+(PCTDA2/CDRMA2))*PLTE))+PLTE),((((CCTDA2−PCTDA2)/PCTDA2)*(1+(PCTDA2/CDRMA2))*PLTE)+PLTE)))))−CLTE)/CLTE)))))/1.3\hat{ }(CCTDA2/CDRMA2))

Modified New Dose Agent 1 (Taxol) (MND1) is calculated by a dose modifier. It is the newly calculated drug dose of Agent 1 (i.e., Taxol) based upon the fit of the drug on its dose response curve, the individualization required for this patient to achieve the desired marker as well as the observed relationship between dose and marker thus determining the relationship as inverse or direct, which equals to,
=IF(E$10=“Yes”,E19−E21,E19+E21)
That is,
=IF(INVERSEMARKER=“Yes”,NAD1−AVA1,NAD1+AVA1)

Modified New Dose Agent 2 (Gemzar) (MND2) is calculated by a dose modifier. It is the newly calculated drug dose of Agent 2 (i.e., Gemzar) based upon the fit of the drug on its dose response curve, the individualization required for this patient to achieve the desired marker as well as the observed relationship between dose and marker thus determining the relationship as inverse or direct, which equals to,
=IF(E$10=“Yes”,F19−F21,F19+F21)
That is,
=IF(INVERSEMARKER=“Yes”,NAD2−AVA2,NAD2+AVA2)

Efficacy/Toxicity Determinant Agent 1 (Taxol) (ETD1) is a value determined by the prescriber. Set at an initial value of 1, this value is then adjusted based upon actual case scenarios with a full data set to adjust the value to identify the contribution to the toxicity or efficacy until the actual value is achieved. It can be used to modify dose based upon non-pharmacologic effects such as diet, exercise and activity as determined by the prescriber.

Efficacy/Toxicity Determinant Agent 2 (Gemzar) (ETD2) is a value determined by the prescriber. Set at an initial value of 1, this value is then adjusted based upon actual case scenarios with a full data set to adjust the value to identify the contribution to the toxicity or efficacy until the actual value is achieved. It can be used to modify dose based upon non-pharmacologic effects such as diet, exercise and activity as determined by the prescriber. A non-pharmacologic two-part combination therapy example could include a cholesterol lowering agent and exercise or cholesterol lowering agent and a restrictive diet. In place of dose the exercise value would be measured in ‘METS’ (Metabolic Equivalent of oxygen consumption) or the dietary prescription in calories or grams of fat or cholesterol.

| NEW DOUBLET | | Lipitor | Aerobics |
|---|---|---|---|
| Previous Cycle Total | | 210 | 140/week |
| Dose/PCTDA1, 2 | | | |
| Current Cycle Total | | 280 | 168/week |
| Dose/CCTDA1, 2 | | | |
| Date of Current Cycle Dose | | Oct. 21, 2004 | Oct. 21, 2004 |
| Cycle Dosing Range Maximum | | 560 | 280 |
| Multiple of Dose Range | | 1 | 1 |
| Maximum | | | |
| Cycle Dosing Range | | 560 | 280 |
| Maximum/CDRMA1, 2 | | | |
| LDL | Inverse MarkerM1 | Yes | |
| | Previous Level of | 150 | |
| | Efficacy/PLTEM1 | | |
| | Current Level of | 120 | |
| | Efficacy/CLTEM1 | | |
| | Date Marker Taken | Dec. 05, 2004 | |
| | Desired Level of | 100 | |
| | Efficacy/DLTEM1 | | |
| | Marker Validity | valid | |
| | New Dose/NAD1, 2 | 340.00 | 210 |
| | Projected | 105 | 105 |
| | MarkerI/PMA1, 2 | | |
| | Adjusted | 20.00 | 35 |
| | Value/AVA1, 2 | | |
| | Modified New | 320.00 | 175 |
| | Dose/MNDA1, 2 | | |

### Non-Pharm Example 1

### Doublet Agent One: Pharmacologic, Agent Two:Non-Pharmacologic (Exercise)

### EXAMPLE 3

Multi-Agent (Three) Cycle Dose Modifier

Taxol, Gemzar and Carboplatin are used in this example as the three agents. Table 3 illustrates a method of revising a drug dose in treating a patient with three pharamcologic and non-pharmacological modalities to optimize pharmacologic outcome, e.g., to reduce risk of an adverse drug event while assessing the clinical relevancy and validity of the measured surrogate markers.

TABLE 3
Chemotherapeutic Multi-Agent Cycle Dose Modifier

| NEW TRIPLET | | Taxol | Gemzar | Carboplatin | |
|---|---|---|---|---|---|
| Previous Cycle Total Dose/PCTDA1, 2, 3 | | 400.00 | 8000.00 | 600.00 | dblPreviousDose A1, A2, A3 |
| Current Cycle Total Dose/CCTDA1, 2, 3 | | 450.00 | 7000.00 | 650.00 | dblCurrentDose A1, A2, A3 |
| Date of Current Cycle Dose | | Oct. 21, 2004 | Oct. 21, 2004 | Oct. 09, 2004 | varDoseDate A1, A2, A3 |
| Cycle Dosing Range Maximum | | 500 | 3300 | 500 | dblMaxRange A1, A2, A3 |
| Multiple of Dose Range Maximum | | 2 | 2 | 2 | intRangeModifier A1, A2, A3 |
| Cycle Dosing Range Maximum/CDRMA1, 2, 3 | | 1000 | 6600 | 1000 | dblRange A1, A2, A3 |
| Current Cycle | Inverse MarkerM1 | Yes | | | bollsInverseMarker M1 |
| | Previous Level of Toxicity/PLTEM1 | 0.40 | | | dblPreviousLevel M1 |
| | Current Level of Toxicity/CLTEM1 | 0.80 | | | dblCurrentLevel M1 |
| | Date Marker Taken | Oct. 30, 2004 | | | varMarkerDate M1 |
| | Desired Level of Toxicity/DLTEM1 | 1.50 | | | dblDesiredLevel M1 |
| | Agent Fraction/FA1, 2, 3 | 0.21 | 0.49 | 0.30 | |
| | Total Share of Effect/TSEA1, 2, 3 | 0.04 | 0.24 | 0.09 | |
| | Proportion of Effect/PEA1, 2, 3 | 0.12 | 0.64 | 0.24 | |
| | Marker Validity | valid | valid | valid | compare 2 dayMarkL_Limit & |
| | | | | | dayMarkU_Limit |
| | New Dose/NAD1, 2, 3 | 418.58 | 5089.22 | 566.77 | |
| | Projected MarkerI/PMA1, 2, 3 | 0.88 | 1.25 | 0.97 | |
| | Adjusted Value/AVA1, 2, 3 | −46.99 | −383.42 | −62.12 | |
| | Modified New Dose/MNDA1, 2, 3 | 465.56 | 5472.65 | 628.89 | |

In Table 3, Previous Cycle Total Dose Agent 1 (PCTD1) is a value of Taxol directly entered without a new calculation. It is the cumulative amount of Taxol administered during the previous treatment cycle as defined by the total amount of drug given over a fixed period of time, the total mg/m² administered throughout the defined regimen cycle period. The cycle is defined when the dose modifier is constructed.

Previous Cycle Total Dose Agent 2 (PCTD2) is a value of Gemzar directly entered without a new calculation. It is the cumulative amount of Gemzar administered during the previous treatment cycle as defined by the total amount of drug given over a fixed period of time, the total mg/m² administered throughout the defined regimen cycle period. The cycle is defined when the dose modifier is constructed.

Previous Cycle Total Dose Agent 3 (PCTD3) is a value of Carboplatin directly entered without a new calculation. It is the cumulative amount of Carboplatin administered during the previous treatment cycle as defined by the total amount of drug given over a fixed period of time, the total mg/m² administered throughout the defined regimen cycle period. The cycle is defined when the dose modifier is constructed.

Current Cycle Total Dose Agent 1 (CCTD1) is a value of Taxol directly entered without a new calculation. It is the proposed amount of Taxol to be given in the current treatment cycle.

Current Cycle Total Dose Agent 2 (CCTD2) is a value of Gemzar directly entered without a new calculation. It is the proposed amount of Gemzar to be given in the current treatment cycle.

Current Cycle Total Dose Agent 3 (CCTD3) is a value of Carboplatin directly entered without a new calculation. It is the proposed amount of Carboplatin to be given in the current treatment cycle.

Date of Current Cycle Dose is based on the initiation of the cycle (in this instance; the cycle was predetermined to be a fourteen day period).

Previous Level of Toxicity-Efficacy (PLTE) is a value directly entered such as from a lab report. It is the numerical value of the marker of toxicity or efficacy reported at the previous dose cycle adjustment (evaluated at a specified elapsed time sufficient to capture the nadir of the dose effect). The surrogate marker is selected when the dose modifier is constructed.

Current Level of Toxicity-Efficacy (CLTE) is a value directly entered such as from a lab report. It is the numerical value of the marker of toxicity or efficacy reported with the current dose cycle. The surrogate marker is that value that illustrates the expression of the toxicity or efficacy of the agent on a specific and pre-selected value determined when the dose modifier is constructed. The value is selected based upon the agent, its pharmacologic properties and the disease condition under treatment. The confidence or validity of the marker is based upon its maturity of effect in that a nadir or cycle of effect is dynamic and may vary based upon the time span between receipt of the agent and sampling of the value. Therefore the value may be valid or invalid based upon this timing factor. If invalid the dose adjustment cannot be made. If valid the value is fully appreciated and the dose modification can be calculated. The surrogate marker is selected when the dose modifier is constructed.

Date Marker Taken is the date that the surrogate marker has been measured.

Desired Level of Toxicity-Efficacy (DLTE) is a value directly entered such as by the prescriber. It is the desired or goal value of the marker of toxicity or efficacy that is to be achieved by the dose modification.

Cycle Dosing Range Maximum Agent 1 (CDRM1) is a value of Taxol determined when the dose modifier is constructed. It is the maximum dosing range of Taxol the patient can receive during the cycle calculated by multiplying the maximum dosing range determined during approval by the number of doses given in each cycle.

Cycle Dosing Range Maximum Agent 2 (CDRM2) is a value of Gemzar determined when the dose modifier is constructed. It is the maximum dosing range of Gemzar the patient can receive during the cycle calculated by multiplying the maximum dosing range determined during approval by the number of doses given in each cycle.

Cycle Dosing Range Maximum Agent 3 (CDRM3) is a value of Carboplatin determined when dose modifier constructed. It is the maximum dosing range of Carboplatin the patient can receive during the cycle calculated by multiplying the maximum dosing range determined during approval by the number of doses given in each cycle.

Fraction of the Dose Agent 1 (FA1) is a value of Taxol calculated by a dose modifier. The Fraction of the Dose Agent 1 (i.e., Taxol) is used to determine the amount of the individual drugs proportion within the combined dose. This number is calculated by dividing the current cycle total dose of the agent by the cycle dose maximum range of the agent divided by the sum of the current cycle total dose of the agent by cycle dose maximum range of the agent and the current cycle total dose of the second agent by cycle dose maximum range of the second agent. Which equals,
=($E4/$E8)/($E4/$E8+$F4/$F8+$G4/$G8)
That is,
=(CCTDA1/CDRMA1)/(CCTDA1/CDRMA1+CCTDA2/CDRMA2+CCTDA3/CDRMA3)

Fraction of the Dose Agent 2 (FA2) is a value of Gemzar calculated by a dose modifier. The Fraction of the Dose Agent 2 (i.e., Gemzar) is used to determine the amount of the individual drugs proportion within the combined dose. This number is calculated by dividing the current cycle total dose of the agent by the cycle dose maximum range of the agent divided by the sum of the current cycle total dose of the agent by cycle dose maximum range of the agent and the current cycle total dose of the second agent by cycle dose maximum range of the second agent. Which equals,
=($F4/$F8)/($E4/$E8+$F4/$F8+$G4/$G8)
That is,
=(CCTDA2/CDRMA2)/(CCTDA1/CDRMA1+CCTDA2/CDRMA2+CCTDA3/CDRMA3)

Fraction of the Dose Agent 3 (FA3) is a value of Carboplatin calculated by a dose modifier. The Fraction of the Dose Agent 3 (i.e., Carboplatin) is used to determine the amount of the individual drugs proportion within the combined dose. This number is calculated by dividing the current cycle total dose of the agent by the cycle dose maximum range of the agent divided by the sum of the current cycle total dose of the agent by cycle dose maximum range of the agent and the current cycle total dose of the second agent by cycle dose maximum range of the second agent. Which equals,
=($G4/$G8)/($E4/$E8+$F4/$F8+$G4/$G8)
That is,
=(CCTDA3/CDRMA3)/(CCTDA1/CDRMA1+CCTDA2/CDRMA2+CCTDA3/CDRMA3)

Total Share of Effect Agent 1 (TSE1) (Taxol) is calculated by a dose modifier. This fraction value is used to describe the percentage of effect attributed to the individual drug within the mix based upon previous calculation using the fraction and the fit to actual experience with the drug mix and marker and the relationship of the drugs effect to the marker. This calculation is reflected on the data sheet and is part of “building” each dose modifier. The determinant of efficacy/toxicity is a combination specific and unique to the individual marker.

Initially based upon the assessment of the agents' individual characteristics, peer reviewed publications and available clinical experience identified the likelihood and degree of positive and negative effects (markers) of all agents in the combination therapy. With this approximation, this value is applied to an existing dataset and correlation, standard deviation and standard error are calculated. The number is then adjusted to derive the optimal determinant value.

The process is repeated for each individual marker, as the value is unique to each individual marker when the dose modifier is constructed. Which equals,
=($E4/$E8)/($E4/$E8+$F4/$F8+$G4/$G8)*E15
That is,
=(CCTDA1/CDRMA1)/(CCTDA1/CDRMA1+CCTDA2/CDRMA2+CCTDA3/CDRMA3)*FA1

Total Share of Effect Agent 2 (Gemzar) (TSE2) is calculated by a dose modifier. This fraction value is used to describe the percentage of effect attributed to the individual drug within the mix based upon previous calculation using the fraction and the fit to actual experience with the drug mix and marker and the relationship of the drugs effect to the marker. This calculation is reflected on the data sheet and is part of “building” each dose modifier. The determinant of efficacy/toxicity is a combination specific and unique to the individual marker.

Initially based upon the assessment of the agents' individual characteristics, peer reviewed publications and available clinical experience identified the likelihood and degree of positive and negative effects (markers) of all agents in the combination therapy. With this approximation, this value is applied to an existing dataset and correlation, standard deviation and standard error are calculated. The number is then adjusted to derive the optimal determinant value.

The process is repeated for each individual marker, as the value is unique to each individual marker when the dose modifier is constructed. Which equals,
=($F4/$F8)/($E4/$E8+$F4/$F8+$G4/$G8)*F15
That is,
=(CCTDA2/CDRMA2)/(CCTDA1/CDRMA1+CCTDA2/CDRMA2+CCTDA3/CDRMA3)*FA2

Total Share of Effect Agent 3 (TSE3) (Carboplatin) is calculated by a dose modifier. This fraction value is used to describe the percentage of effect attributed to the individual drug within the mix based upon previous calculation using the fraction and the fit to actual experience with the drug mix and marker and the relationship of the drugs effect to the marker. This calculation is reflected on the data sheet and is part of “building” each dose modifier. The determinant of efficacy/toxicity is a combination specific and unique to the individual marker.

Initially based upon the assessment of the agents' individual characteristics, peer reviewed publications and available clinical experience identified the likelihood and degree of positive and negative effects (markers) of all agents in the combination therapy. With this approximation, this value is applied to an existing dataset and correlation, standard deviation and standard error are calculated. The number is then adjusted to derive the optimal determinant value.

The process is repeated for each individual marker, as the value is unique to each individual marker when the dose modifier is constructed. Which equals,
=($G4/$G8)/($E4/$E8+$F4/$F8+$G4/$G8)*G15
That is,
=(CCTDA3/CDRMA3)/(CCTDA1/CDRMA1+CCTDA2/CDRMA2+CCTDA3/CDRMA3)*FA3

Proportion of Effect 1 (Agent 1-Taxol) (PEI) is calculated by a dose modifier. Once the total share of effect is determined, then each individual agent's share of that effect is divided by the total effect to determine what proportion of the change in the marker is that of the individual agent. PE1 equals to the total share of the affect of the first agent divided by the total share of affect for agent one plus the total share of affect for agent two plus the total share of affect for agent three. Which equals,
=E16/(E16+F16+G16)
That is,
=TSEA1/(TSEA1+TSEA2+TSEA3)

Proportion of Effect 2 (Agent 2-Gemzar) (PE2) is calculated by a dose modifier. Once the total share of effect is determined, then each individual agent's share of that effect is divided by the total effect to determine what proportion of the change in the marker is that of the individual agent. PE2 equals to the total share of the affect of the first agent divided by the total share of affect of agent one plus the total share of affect for agent two plus the total share of affect for agent three. Which equals,
=F16/(E16+F16+G16)
That is,
=TSEA2/(TSEA1+TSEA2+TSEA3)

Proportion of Effect 3 (Agent 3-Carboplatin) (PE3) is calculated by a dose modifier. Once the total share of effect is determined, then each individual agent's share of that effect is divided by the total effect to determine what proportion of the change in the marker is that of the individual agent. PE3 equals to the total share of the affect of the first agent divided by the total share of affect of agent one plus the total share of affect for agent two plus the total share of affect for agent three. Which equals,
=G16/(E16+F16+G16)
That is,
=TSEA3/(TSEA1+TSEA2+TSEA3)

Marker Validity is a response recorded in the cell as valid or invalid.

| | Marker: | | | | Neutrophil | | Hemoglobin | | | |
|---|---|---|---|---|---|---|---|---|---|---|
| | Max | | Platelets | | Count (ANC) | | (Hgb) | | INR | |
| Agent | Dose | Inverse | LL | UL | LL | UL | LL | UL | LL | UL |
| Carboplatin | 500 | yes | 17 | 25 | 17 | 25 | 14 | 21 | | |
| Cisplatin | 220 | yes | 18 | 23 | 18 | 23 | 18 | 23 | | |
| Gemzar | 3300 | yes | 7 | 10 | 7 | 10 | 7 | 10 | | |
| Taxol | 500 | yes | 8 | 11 | 8 | 11 | 8 | 11 | | |
| Taxotere | 500 | yes | 6 | 8 | 6 | 8 | 8 | 11 | | |
| Warfarin Sodium | 160 | no | | | | | | | 4 | 5 |

A lookup table has been constructed (see above example), in the form of a table, to represent the relationship of each trial Agent and its qualified Clinical Markers. This table includes information regarding the Maximum Dose and the Inverse Marker Applicability for each Agent. The gray cells on the Cycle Dose Modifier indicate these values.

The table also includes information regarding the nadir of applicability for each Agent—Marker combination. The range, in days and indicated as the Lower Limit and Upper limit, has been determined by calculating a percentage (20%) above and below the true nadir value when only a single value is known. Otherwise, the upper and lower nadir values are used. The relationship of Agents to Markers can best be exhibited by the following Entity Relationship Diagram, to-wit: 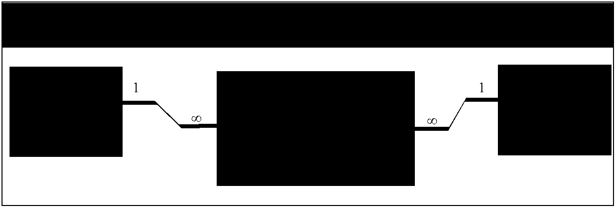
Please note that the relationship of Agents to Associations is one-to-many as is the relationship of Markers to Associations. The values of Upper and Lower limits is shown as a property of the Association between Markers and Agents. The Relevancy of a Marker can be computed using the lookup table by comparing the number of days elapsed between the date of the application of the current dose and the date on which the Marker was taken to the upper and lower values for that Agent Marker Association. If the elapsed days are greater than or equal to the lower limit and less than or equal to the upper limit, then the calculations provided by the Cycle Dose Modifier are said to be relevant and thereby applicable as indicated in the yellow Marker Validity cell. The Relevancy of Cycle Dose Modifier Calculations for Agent 1 is based on the Validity of Marker for Agent 1 calculated by:
=IF(AND((($E13−E5)>=VLOOKUP(E$2,Validity!$A$3:$K$8,6)),((E$13−E5)<=VLOOKUP(E$2,Validity!$A$3:$K$8,7))) ,“valid”,“invalid”)
The Relevancy of Cycle Dose Modifier Calculations for Agent 2 is based on the Validity of Marker for Agent 2 calculated by:
=IF(AND((($E13−F5)>=VLOOKUP(F$2,Validity!$A$3:$K$8,6)),((F$13−F5)<=VLOOKUP(F$2,Validity!$A$3:$K$8,7))),“valid”,“invalid”)
The Relevancy of Cycle Dose Modifier Calculations for Agent 3 is based on the Validity of Marker for Agent 3 calculated by:
=IF(AND((($E13−G5)>=VLOOKUP(G$2,Validity!$A$3:$K$8,6)),(($E13−G5)<=VLOOKUP(G$2,Validity!$A$3:$K$8,7))),“valid”,“invalid”)

New Agent Dose 1 (Agent 1-Taxol) (NAD1) is calculated by a dose modifier. The new cycle dose of Agent 1 (i.e., Taxol) is based upon the plane identity of the agent as well as the observed relationship between dose and marker and determining the relationship as inverse or direct, which equals,
=IF(E$10=“yes”, $E4+((E$12−E$14)/E$12)*E17/(1+($E4/$E8))*$E4,$E4−((E$12−E$14)/E$12)*E17/(1+($E4/$E8))*$E4)
That is,
=IF(InverseMarker=“yes”, CCTDA1+((CLTE−DLTE)/CLTE)*PEA1/(1+(CCTDA1/CDRMA1))*CCTDA1,CCTDA1−((CLTE−DLTE)/CLTE)*PEA1/(1+(CCTDA1/CDRMA1))*CCTDA1)

New Agent Dose 2 (Agent 2-Gemzar) (NAD2) is calculated by a dose modifier. The new cycle dose of Agent 2 (i.e., Gemzar) is based upon the plane identity of the agent as well as the observed relationship between dose and marker and determining the relationship as inverse or direct, which equals,
=IF(E$10=“yes”, $F4+((E$12−E$14)/E$12)*F17/(1+($F4/$F8))*$F4,$F4−((E$12−E$14)/E$12)*F17/(1+($F4/$F8))*$F4)
That is,
=IF(InverseMarker=“yes”, CCTDA2+((CLTE−DLTE)/CLTE)*PEA2/(1+(CCTDA2/CDRMA2))*CCTDA2,CCTDA2−((CLTE−DLTE)/CLTE)*PEA2/(1+(CCTDA2/CDRMA2))*CCTDA2)

New Agent Dose 3 (Agent 3-Carboplatin) (NAD3) is calculated by a dose modifier. The new cycle dose of Agent 3 (i.e., Carboplatin) is based upon the plane identity of the agent as well as the observed relationship between dose and marker and determining the relationship as inverse or direct, which equals,
=IF(E$10=“yes”, $G4+((E$12−E$14)/E$12)*G17/(1+($G4/$G8))*$G4,$G4−((E$12−E$14)/E$12)*G17/(1+($G4/$G8))*$G4)
That is,
=IF(InverseMarker=“yes”, CCTDA3+((CLTE−DLTE)/CLTE)*PEA3/(1+(CCTDA3/CDRMA3))*CCTDA3,CCTDA3−((CLTE−DLTE)/CLTE)*PEA3/(1+(CCTDA3/CDRMA3))*CCTDA3)

Projected Marker Agent 1 (Taxol) (PMA1) is calculated by a dose modifier. The expected marker value for Agent 1 is based upon the new dose calculation. Which equals,
=IF(OR(and(E$10=“YES”,E22=(2*$E4))),−1*((((((E22)−$E4)/$E4)*(1+(($E4/$E8))*1))*E$12)−E$12),IF(OR(and(E$10=“YES”,E22=(0.5*$E4))),−1*((((((E22)−$E4)/$E4)*(1+(($E4/$E8))*1))*E$12)−E$12),IF(E$10=“YES”,−1((((((E22+E21)−$E4)/$E4)*(1+(($E4/$E8))*1))*E$12)−E$12)IF(or(and(E$10=“NO”,E22=(2*$E4))),((((E22−$E4)/$E4)*(1+(($E4$E8))*1))*E$12,IF(or)and(es10=“NO”,E22=(0.5*$e4))),((((E22−$E4)/$E4)*(1+((sE4/$E8))*1))*E$12)+E$12,(((((E22−E21)−$E4)/$E4)*(1+(($E4/sE8))*1))*Es12)+E$12)))))
That is,
=IF(OR(and(InverseMarker=“YES”,MNDA1=(2*CCTDA1))),−1*((((((MNDA1)−CCTDA1)/CCTDA1)*(1+((CCTDA1/CDRMA1))*1))*CLTE)−CLTE),IF(OR(and(InverseMarker=“YES”,MNDA1=(0.5*CCTDA1))),−1*((((((MNDA1)−CCTDA1)/CCTDA1)*(1+((CCTDA1/CDRMA1))*1))*CLTE)−CLTE),IF(InverseMarker=“YES”,−1*((((((MNDA1+AVA1)−CCTDA1)/CCTDA1)*(1+((CCTDA1/CDRMA1))*1))*CLTE)−CLTE),IF(OR(and(InverseMarker=“NO”,MNDA1=(2* CCTDA1))),((((MNDA1−CCTDA1)/CCTDA1)*(1+((CCTDA1/CDRMA1))*1))*CLTE)+CLTE,IF(OR(and(InverseMarker=“NO”,MNDA1=(0.5*CCTDA1))),((((MNDA1−CCTDA1)/CCTDA1)*(1+((CCTDA1/CDRMA1))*1))*CLTE)+CLTE,(((((MNDA1−AVA1)−CCTDA1)/CCTDA1)*(1+((CCTDA1/CDRMA1))*1))*CLTE)+CLTE)))))

Projected Marker Agent 2 (Gemzar) (PM2) is calculated by a dose modifier. The expected marker value for Agent 2 is based upon the new dose calculation. Which equals,
=IF(OR(and(E$10=“YES”,F22=(2*$F4))),−1*((((((F22)−$F4)/$F4)*(1+(($F4/$F8))*1))*E$12)−E$12),IF(OR(and(E$10=“YES”,F22=(0.5*$F4))),−1*((((((F22)−$F4)/$F4)*(1+(($F4/$F8))*1))*E$12)−E$12),IF(E$10=“YES”,−1*((((((F22+F21)−$F4)/$F4)*(1+(($F4/$F8))*1))*E$12)−E$12),IF(OR(and(E$10=“NO”,F22=(2*$F4))),((((F22−$F4)/$F4)*(1+(($F4/$F8))*1))*E$12)+E$12,IF(OR(and(E$10=“NO”, F22=(0.5*$F4))),((((F22−$F4)/$F4)*(1+(($F4/$F8))*1))*E$12)+E$12,(((((F22−F21)−$F4)/$F4)*(1+(($F4/$F8))*1))*E$12)+E$12)))))
That is,
=IF(OR(and(InverseMarker=“YES”,MNDA2=(2*CCTDA2))),−1*((((((MNDA2)−CCTDA2)/CCTDA2)*(1+((CCTDA2/CDRMA2))*1))*CLTE)−CLTE), IF(OR(and(InverseMarker=“YES”,MNDA2=(0.5*CCTDA2))),−1*((((((MNDA2)−CCTDA2)/CCTDA2)*(1+((CCTDA2/CDRMA2))*1))*CLTE)−CLTE),IF(InverseMarker=“YES”,−1*((((((MNDA2+AVA2)−CCTDA2)/CCTDA2)*(1+((CCTDA2/CDRMA2))*1))*CLTE)−CLTE),IF(OR(and(InverseMarker=“NO”,MNDA2=(2*CCTDA2))),((((MNDA2−CCTDA2)/CCTDA2)*(1+((CCTDA2/CDRMA2))*1))*CLTE)+CLTE,IF(OR(and(InverseMarker=“NO”,MNDA2=(0.5*CCTDA2))),((((MNDA2−CCTDA2)/CCTDA2)*(1+((CCTDA2/CDRMA2))*1))*CLTE)+CLTE,(((((MNDA2−AVA2)−CCTDA2)/CCTDA2)*(1+((CCTDA2/CDRMA2))*1))*CLTE)+CLTE)))))

Projected Marker Agent 3 (Carboplatin) (PMA3) is calculated by a dose modifier. The expected marker value for Agent 3 is based upon the new dose calculation. Which equals,
=IF(OR(and(E$10=“YES”, G22=(2*$G4))),−1*((((((G22)−$G4)/$G4)*(1+(($G4/$G8))*1))*E$12)−E$12), IF(OR(and(E$10=“YES”,G22=(0.5*$G4))),−1*((((((G22)−$G4)/$G4)*(1+(($G4/$G8))*1))*E$12)−E$12),IF(E$10=“YES”,−1*((((((G22+G21)−$G4)/$G4)*(1+(($G4/$G8))*1))*E$12)−E$12),IF(OR(and(E$10=“NO”,G22=(2*$G4))),((((G22−$G4)/$G4)*(1+(($G4/$G8))*1))*E$12)+E$12,IF(OR(and(E$10=“NO”,G22=(0.5*$G4))),((((G22−$G4)/$G4)*(1+(($G4/$G8))*1))*E$12)+E$12,(((((G22−G21)−$G4)/$G4)*(1+(($G4/$G8))*1))*E$12)+E$12)))))
That is,
=IF(OR(and(InverseMarker=“YES”,MNDA3=(2*CCTDA3))),−1*((((((MNDA3)−CCTDA3)/CCTDA3)*(1+((CCTDA3/CDRMA3))*1))*CLTE)−CLTE),IF(OR(and(InverseMarker=“YES”,MNDA3=(0.5*CCTDA3))),−1*((((((MNDA3)−CCTDA3)/CCTDA3)*(1+((CCTDA3/CDRMA3))*1))*CLTE)−CLTE), IF(InverseMarker=“YES”,−1*((((((MNDA3+AVA3)−CCTDA3)/CCTDA3)*(1+((CCTDA3/CDRMA3))*1))*CLTE)−CLTE),IF(OR(and(InverseMarker=“NO”,MNDA3=(2*CCTDA3))),((((MNDA3CCTDA3)/CCTDA3)*(1+((CCTDA3/CDRMA3))*1))*CLTE)+CLTE, IF(OR(and(InverseMarker=“NO”,MNDA3=(0.5*CCTDA3))),((((MNDA3−CCTDA3)/CCTDA3)*(1+((CCTDA3/CDRMA3))*1))*CLTE)+CLTE,(((((MNDA3−AVA3)−CCTDA3)/CCTDA3)*(1+((CCTDA3/CDRMA3))*1))*CLTE)+CLTE)))))

Adjusted Value Agent 1 (Taxol) (AVA1) is calculated by a dose modifier. It is the stochastic open loop designed to adjust the dose (Agent 1, i.e., Taxol) based upon the individual's actual response versus the expected response, which may increase the dose, reduce the dose or leave the dose the same. The amount of the dose that is reserved is Variable Sensitivity Value (VSV), which is determined by the pharmacologic properties of the agent when the dose modifier is built. Which equals,
=(IF(OR(and((E$12<E$14),and((IF(E$11=0,0,IF($E3=0,0,IF(E$10=“Yes”,(−1*(((($E4−$E3)/$E3)*(1+($E3/$E8))*E$11))+E$11),(((($E4−$E3)/$E3)*(1+($E3/$E8))*E$11)+E$11)))))>E$12))),((20/100)*$E4)*(((IF(E$11=0,0,IF($E3=0,0IF (E$10=“Yes”,(−1*(((($E4=$E3)/$e3)*(1+(SE3/$e8))*ES11))+E$11),(((($E4−$E3)/$E3)*(1+($E3/$E8))*E$11)+E$11)))))−E$12)/E$12)IF(E$12<E$12,(−20/100*$E4)*((E$12−(IF(E$11=0,0,IF($E3=0,0,IF(E$10=“Yes”,(−1*(((($E4−$E3)$E3)*(1+(RE3/$E8))*E$11))+E$11),(((($E4−$E3)$E3)*(1+($E3/$E8))*E$11)+E$11)))))/(E$12)IF((IF(E$11=0,0,IF($E3=0,0IF(E$10=“Yes”,(−1+(((($E4−$E3)/$E3)*(1+($E3/$E8))*E$11))+E$11),(((($E4−$E3)/$E3)*(1+($E3$E8))*E$11)+E$11)))))>E$12),(−(20/100)*$E4)*((E$12−(IF(E$11=0,0IF($E3=0,0IF(E$10=“Yes”,(−1*(((($E4−$E3)/$E3)*(1+($E3/$E8))*E$11))+E$11)(((($e4−$E3)/$E3)*(1+($E3/$E8))*E$11)+E$11)))))/E$12),((10/100)*$e4)*(((IF(e$11=0,0,IF($E3=0,0IF(E$10=“Yes”,(−1*(((($E4−$E3)*(1+($E3/$E8))*E$11))+E$11),(((($E4−$E3)$E3)*(1+($E3/$E8))*E$11)+E$11)))))−E$12)/E$12)))))/1.3\hat{ }($E4/$E8))
That is,
=(IF(OR(and((CLTE<DLTE),and((IF(PLTE=0,0,IF(PCTDA1=0,0,IF(InverseMarker=“Yes”,(−1*((((CCTDA1−PCTDA1)/PCTDA1)*(1+(PCTDA1/CDRMA1))*PLTE))+PLTE),((((CCTDA1−PCTDA1)/PCTDA1)*(1+(PCTDA1/CDRMA1))*PLTE)+PLTE)))))>CLTE))),((20/100)*CCTDA1)*(((IF(PLTE=0,0,IF(PCTDA1=0,0,IF(InverseMarker=“Yes”,(−1*((((CCTDA1−PCTDA1)/PCTDA1)*(1+(PCTDA1CDRMA1)*PLTE))+PLTE),((((CFTDA1−PCTDA1)/PCTDA1)*(1+(PCTDA1/CDRMA1))+PLTE)+PLTE)))))>CLTE))),((20/100)*CCTDA1)*(((IF(PLTE=0,0IF(PCTDA1=0,0IF(InverseMarker=“Yes”,(−1*((((CCTDA1−PCTDA1)/PCTDA1)*(1+(PCTDA1/CDRMA1))*PLTE))+PLTE),((((CCTDA1−PCTDA1)/PCTDA1)*(1+(PCTDA1/CDRMA1))*PLTE)+PLTE)))))−CLTE)/CLTE),IF(CLTE<DLTE,(−20/100*CCTDA1)−PCTDA1)/PTDA1)*(1+(PCTDA1/CDRMA1))*PLTE))+PLTE),((((CCTDA1−PCTDA1)/PCTDA1)*(1+(PCTDA1/CDRMA1))*PLTE)+PLTE))))))/CLTE),IF((IF(PLTE=0,0IF(PCT DA1=0,0IF(InverseMarker=“Yes”,(−1*((((CCTDA1−PCTDA1)/PCTDA1)*(1+(PCTDA1/CDRMA1))*PLTE))+PLTE),((((CCTDA1−PCTDA1)PCTDA1)*(1+(CTDA1/CDRMA1))*PLTE)+PLTE)))))>CLTE,(−(20/100)*CCTDA1)*((CLTE−(IF(PLTE=0,0,IF(PCTDA1=0,0IF(InverseMarker=“Yes”,(−1*((((CCTDA−PCTDA1)/CTDA1)*(1+(PCTDA1/CDRMA1))*PLTE))+PLTE),((((CCTDA1−PCTDA1)/CTDA1)*(1+(PCTDA1/CDRMA1))*PLTE)+PLTE))))))/CLTE),((20/100)*CCTDA1)*(((IF (PLTE=0,0IF(PCTDA1=0,0,IF(InverseMarker=“Yes”,(−1*((((CCTDA1−PCTDA1)/PCTDA1)*(1+(PCTDA1/CDRMA1))*PLTE))+PLTE),((((CCTDA1−PCTDA1)/PCTDA1)*(1+(PCTDA1/CDRMA1))*PLTE)+PLTE)))))−CLTE)/CLTE)))))/(1.3\mathring{}(CCTDA1/CDRMA1))

Adjusted Value Agent 2 (Gemzar) (AVA2) is calculated by a dose modifier. It is the stochastic open loop designed to adjust the dose (Agent 2, i.e., Gemzar) based upon the individual's actual response versus the expected response, which may increase the dose, reduce the dose or leave the dose the same. The amount of the dose that is reserved is Variable Sensitivity Value (VSV), which is determined by the pharmacologic properties of the agent when the dose modifier is built. Which equals,
=(IF(OR(and((E$12<E$14),and((IF(E$11=0,0,IF($F3=0,0,IF(E$10=“Yes”,(−1*(((($F4−$F3)/$F3)*(1+($F3/$F8))*E$11))+E$11),(((($F4−$F3)/$F3)*(1+($F3/$F8))*E$11)+E$11)))))>E$12))),((20/100)*$F4)*(((IF(E$11=0,0,IF($F3,=0,0IF(E$10=“Yes+,(−1*(((($F4−FF3)/$F3)*(1+($F3/$F8))*E$11))+E$11),(((($F4−$F3)/$F3)*(1+($F3/$F8))*E$11)+E$11)))))−E$12)/E$12), IF(E$12<E$14,(−20/100*FF4)*((E$12−(IF(E$11=0,0,IF($F3=0,0,IF(E$10=“Yes+,(−1*(((($F4−FF3)/$F3)*(1+($F3/$F8))*E$11))+E$11),(((($F4−$F3)/$F3)*(1+($F3/$F8))*E$11)+E$11))))))/E$12),IF((IF(E$11=0,0,IF($F3=0,0,IF(E$10=“Yes”,(−1*(((($F4−$F3)/$F3)*1+($F3/$F8))*E$11))+E$11),(((($F4−$F3)/$F3)*(1+($F3/$F8))*E$11)+E$11)))))>E$12,(−(20/100)*$F4)*((E$12−(IF(E$11=0,0IF($F3=0,0,IF(E$10=“Yes”,*(−1*(((($F4−$F3)/$F3)*(1+($F3/$F8))*E$11))+E$11),(((($F4−$F3)/$F3)*(1+(FF3$F8))*E$11)+E$11))))))/E$12),((20/100)*$F4)*(((IF(E$11=0,0IF($F3=0,0IF(E $10=“Yes”,(−1*(((($F4−$F3)*(1+($F3/$F8))*E$11))+E$11),(((($F4−$F3/$F3)*(1+(FF3/$F8))*ES11)+E$11))))−E$12)/E$12)))))/(\hat{ }($F4/$F8))
That is,
=(IF(OR(and((CLTE<DLTE),and((IF(PLTE=0,0, IF(PCTDA2=0,0,IF(InverseMarker=“Yes”,(−1*((((CCTDA2−PCTDA2)/PCTDA2)*(1+(PCTDA2/CDRMA2))*PLTE))+PLTE),((((CCTDA2−PCTDA2)/PCTDA2)*(1+(PCTDA2/CDRMA2))*PLTE)+PLTE)))))>CLTE))),((20/100)*CCTDA2)*(((IF(PLTE=0,0,IF(PCTDA2=0,0, IF(InverseMarker=“Yes”,(−1*((((CCTDA2−PCTDA2)/PCTDA2)*(1+(PCTDA2/CDRMA2))*PLTE)+PLTE),((((CCTDA2−PCTDA2)/PCTDA2)*(1+(PCTDA2/CDRMA2))*PLTE)+PLTE)))))−CLTE)/CLTE),IF(CLTE<DLTE,(−20/100*CCTDA2)*((CLTE−(IF(PLTE=0,0,IF(PCTDA2=0,0,IF(InverseMarker=“Yes”,(−1*((((CCTDA2−PCTDA2)/PCTDA2)*(CDRMA2))*PLTE),((((CCTDA2−PCTDA2)/PCTDA2)*(1+(PCTDA2/CDRMA2))*PLTE)+PLTE))))))/CLTE),IF(IF(PLTE=0,0,IF(PCT DA2=0,0IF*(InverseMarker=“Yes”,(−1*((((CCTDA2−PCTDA2)/PCTDA2)(1+(PCTDA2/CDRMA2))*PLTE))+PLTE),((((CCTDA2−PCTDA2)/PCTDA2)*(1+(PCTDA2/CDRMA2))*PLTE)+PLTE)))))>CLTE,(−(20/100)*CCTDA2)*((CLTE−(IF(PLTE=0,0,IF(PCTDA2=0,0IF(InverseMarker=“Yes”,(−1*((((CCTDA2−PCXTDA2)/PCTDA2)PCTDA2)*(1+(PCTDA2/CDRMA2))*PLTE))+PLTE))+PLTE),((((CCTDA2−PCTDA2)/PCTDA2)*(1+(PCTDA2/CDRMA2))*PlTE)+PLTE))))))/CLTE),((20,100)*CCTDA2)*(((IF (PLTE=0,0,IF(PCTDA2=0,0IF(InverseMarker=“Yes”,(−1*((((CCTDA2−(PLTE=0,0,IF(PCTDA2=0,0,IF(InverseMarker=“Yes”,(−1*((((CCTDA2−PCTDA2)/PCTDA2)*(1+(PCTDA2/CDRMA2))*PLTE))+PLTE),((((CCTDA2−PCTDA2)/PCTDA2)*(1+(PCTDA2/CDRMA2))*PLTE)+PLTE)))))−CLTE)/CLTE)))))/(1.3\hat{ }(CCTDA2/CDRMA2))

Adjusted Value Agent 3 (Carboplatin) (AVA3) is calculated by a dose modifier. It is the stochastic open loop designed to adjust the dose (Agent 3, i.e., Carboplatin) based upon the individual's actual response versus the expected response, which may increase the dose, reduce the dose or leave the dose the same. The amount of the dose that is reserved is Variable Sensitivity Value (VSV), which is determined by the pharmacologic properties of the agent when the dose modifier is built. Which equals,
=(IF(OR(and((E$12<E$14),and((IF(E$11=0,0,IF($G3=0,0,IF(E$10=“Yes”,(−1*(((($G4−$G3)/$G3)*(1+($G3/$G8))*E$11))+E$11),(((($G4−$G3)/$G3)*(1+($G3/$G8))*E$11)+E$11)))))>E$12))),((20/100)*$G4)*(((IF(E$11=0,0,IF($G3=0,0,IF(E$10=“Yes”,(−1*(((($G4−$G3)/$G3)*(1+($G3/$G8))*E$11))+E$11),(((($G4−$G3)/$G3)*(1+($G3/$G8))*E$11)+E$11)))))−E$12)/E$12),IF(E$12<E$14,(−20/100*$G4)*((E$12−(IF(E$11=0,0,IF($G3=0,0,IF(E$10=“Yes”,(−1*(((($G4−$G3)/$G3)*(1+($G3/$G8))*E$11))+E$11),(((($G4−$G3)/$G3)*(1+($G3/$G8))*E$11)+E$11))))))/E$12), IF((IF(E$11=0,0, IF($G3=0,0,IF(E$10=“Yes”,(−1*(((($G4−$G3)/$G3)*(1+($G3/$G8))*E$11))+E$11),(((($G4−$G3)/$G3)*(1+($G3/$G8))*E$11)+E$11)))))>E$12,(−(20/100)*$G4)*((E$12−(IF(E$11=0,0,IF($G3=0,0,IF(E$10=“Yes”,(−1*(((($G4−$G3)/$G3)*(1+($G3/$G8))*E$11))+E$11),(((($G4−$G3)/$G3)*(1+($G3/$G8))*E$11)+E$11)))))))/E$12),((20/100)*$G4)*(((IF(E$11=0,0,IF($G3=0,0,IF(E$10=“Yes”,(−1*(((($G4−$G3)/$G3)*(1+($G3/$G8))*E$11))+E$11),(((($G4−$G3)/$G3)*(1+($G3/$G8))*E$11)+E$11)))))))−E$12)/E$12))))))/(1.3\hat{ }($G4/$G8))
That is,
=(IF(OR(and((CLTE<DLTE),and((IF(PLTE=0,0, IF(PCTDA3=0,0,IF(InverseMarker=“Yes”,(−1*((((CCTDA3−PCTDA3)/PCTDA3)*(1+(PCTDA3/CDRMA3))*PLTE))+PLTE),((((CCTDA3−PCTDA3)/PCTDA3)*(1+(PCTDA3/CDRMA3))*PLTE)+PLTE)))))>CLTE))),((20/100)*CCTDA3)*(((IF(PLTE=0,0,IF(PCTDA3=0,0,IF(InverseMarker=“Yes”, (−1*((((CCTDA3−PCTDA3)/PCTDA3)*(1+(PCTDA3/CDRMA3))*PLTE))+PLTE),((((CCTDA3−PCTDA3)/PCTDA3)*(1+(PCTDA3/CDRMA3))*PLTE)+PLTE)))))−PLTE)/CLTE),IF(CLTE<DLTE,(−20/100*CCTDA3)*((CLTE−(IF(PLTE=0,0,IF(PCTDA3=0,0,IF(InverseMarker=“Yes”,(−1*((((CCTDA3−PCTDA3)/PCTDA3)*(1+(PCTDA3/CDRMA3))*PLTE))+PLTE),((((CCTDA3−PCTDA3)/PCTDA3)*(1+(PCTDA3/CDRMA3))*PLTE)+PLTE))))))/CLTE),IF((IF(PLTE=0,0,IF(PCTDA3=0,0,IF(InverseMarker=“Yes”,(−1*((((CCTDA3−PCTDA3)/PCTDA3)*(1+(PCTDA3/CDRMA3))*PLTE))+PLTE),((((CCTDA3−PCTDA3)/PCTDA3)*(1+(PCTDA3/CDRMA3))*PLTE)+PLTE)))))>CLTE,(−(20/100)*CCTDA3)*((CLTE−(IF(PLTE=0,0,IF(PCTDA3=0,0,IF(InverseMarker=“Yes”,(−1*((((CCTDA3−PCTDA3)/PCTDA3)*(1+(PCTDA3/CDRMA3))*PLTE))+PLTE),((((CCTDA3−PCTDA3)/PCTDA3)*(1+(PCTDA3/CDRMA3))*PLTE)+PLTE))))))/CLTE),((20/100)*CCTDA3)*(((IF(PLTE=0,0,IF(PCTDA3=0,0, IF(InverseMarker=“Yes”,(−1*((((CCTDA3−PCTDA3)/PCTDA3)*(1+(PCTDA3/CDRMA3))*PLTE))+PLTE),((((CCTDA3−PCTDA3)/PCTDA3)*(1+(PCTDA3/CDRMA3))*PLTE)+PLTE)))))−CLTE)/CLTE)))))/(1.3\hat{ }(CCTDA3/CDRMA3))

Modified New Dose Agent 1 (Taxol) (MND1) is calculated by a dose modifier. The newly calculated drug dose of Taxol is based upon the fit of the drug on its dose response curve, the individualization required for this patient to achieve the desired marker as well as the observed relationship between dose and marker thus determining the relationship as inverse or direct, which equals to,
=IF(E$10=“Yes”,E19−E21,E19+E21)
That is,
=IF(InverseMarker=“Yes”,NAD1−AVA1, NAD1+AVA1)

Modified New Dose Agent 2 (Gemzar) (MND2) is calculated by a dose modifier. The newly calculated drug dose of Gemzar is based upon the fit of the drug on its dose response curve, the individualization required for this patient to achieve the desired marker as well as the observed relationship between dose and marker thus determining the relationship as inverse or direct, which equals to,
=IF(E$10=“Yes”,F19−F21,F19+F21)
That is,
=IF(InverseMarker=“Yes”,NAD2−AVA2, NAD2+AVA2)

Modified New Dose Agent 3 (Carboplatin) (MND3) is calculated by a dose modifier. The newly calculated drug dose of Carboplatin is based upon the fit of the drug on its dose response curve, the individualization required for this patient to achieve the desired marker as well as the observed relationship between dose and marker thus determining the relationship as inverse or direct, which equals to,
=IF(E$10=“Yes”,G19−G21,G19+G21)
That is,
=IF(InverseMarker=“Yes”,NAD3−AVA3,NAD3+AVA3)

Efficacy/Toxicity Determinant Agent 1 (Taxol) (ETD1) is a value determined by the prescriber. Set at an initial value of 1, this value is then adjusted based upon actual case scenarios with a full data set to adjust the value to identify the contribution to the toxicity or efficacy until the actual value is achieved. It can be used to modify dose based upon non-pharmacologic effects such as diet, exercise and activity as determined by the prescriber.

Efficacy/Toxicity Determinant Agent 2 (Gemzar) (ETD2) is a value determined by the prescriber. Set at an initial value of 1, this value is then adjusted based upon actual case scenarios with a full data set to adjust the value to identify the contribution to the toxicity or efficacy until the actual value is achieved. It can be used to modify dose based upon non-pharmacologic effects such as diet, exercise and activity as determined by the prescriber.

Efficacy/Toxicity Determinant Agent 3 (Carboplatin) (ETD3) is a value determined by the prescriber. Set at an initial value of 1, this value is then adjusted based upon actual case scenarios with a full data set to adjust the value to identify the contribution to the toxicity or efficacy until the actual value is achieved. It can be used to modify dose based upon non-pharmacologic effects such as diet, exercise and activity as determined by the prescriber. A non-pharmacologic three-part combination therapy example could include a cholesterol lowering agent, exercise and a restrictive diet. In place of dose the exercise value would be measured in ‘METS’ (Metabolic Equivalent of oxygen consumption) or the dietary prescription in calories or grams of cholesterol.

| NEW TRIPLET | | Lipitor | Aerobics | Diet |
|---|---|---|---|---|
| Previous Cycle Total | | 220.00 | 140/week | 210/week |
| Dose/PCTDA1, 2, 3 | | | | |
| Current Cycle Total | | 300.00 | 168/week | 182/week |
| Dose/CCTDA1, 2, 3 | | | | |
| Date of Current Cycle Dose | | Oct. 21, | Oct. 21, | Oct. 21, |
| | | 2004 | 2004 | 2004 |
| Cycle Dosing Range Maximum | | 560 | 280 | 210 |
| Multiple of Dose Range | | 1 | 1 | 1 |
| Maximum | | | | |
| Cycle Dosing Range | | 560 | 280 | 210 |
| Maximum/CDRMA1, 2, 3 | | | | |
| LDL | Inverse MarkerM1 | | Yes | |
| | Previous Level | | 140 | |
| | of/PLTEM1 | | | |
| | Current Level of | | 122 | |
| | Efficacy CLTEM1 | | | |
| | Date Marker Taken | | Dec. 05, | |
| | | | 2004 | |
| | Desired Level of | | 100 | |
| | Efficacy/DLTEM1 | | | |
| | Marker Validity | valid | | |
| | New Dose/NAD1, 2, 3 | 340.00 | 210.00 | 140.00 |
| | Projected MarkerI/ | 105 | 105 | 105 |
| | PMA1, 2, 3 | | | |
| | Adjusted Value/ | 20.00 | −35.00 | −35.00 |
| | AVA1, 2, 3 | | | |
| | Modified New Dose/ | 360.00 | 245.00 | 175.00 |
| | MNDA1, 2, 3 | | | |

### Non-Pharm Example 2

### Triplet, Agent One: Pharmacologic, Agent Two: Non-Pharmacologic (Exercise), Agent Three: Diet (Grams of Cholesterol)

Still another embodiment of the present invention advantageously provides a program product or software to revise a cycle dose of at least one agent in a therapy for a patient receiving such a therapy. This program product includes means for accepting as a first input the patient's cycle dose(s) of the at least one agent, means for accepting as a second input determination of the relevancy of at least one surrogate marker indicating a pharmacologic response of the patient, and means responsive to the first input and second input for calculating a revised cycle dose of the at least one agent.

Still yet another embodiment of the present invention advantageously provides a program product or software to revise a cycle dose of at least one agent in a therapy for a patient receiving such a therapy. This program product, for example, includes means for accepting as a first input the patient's cycle dose(s) of the at least one agent, means for accepting as a second input at least one non-pharmacologic modality received by the patient, means for accepting as a third input determination of the relevancy of at least one surrogate marker indicating a pharmacologic response of the patient, means for accepting as a fourth input determination of the relevancy of the at least one non-pharmacologic modality indicating achievement of benefit, and means for determining a revised cycle dose of the at least one agent as a function of the first input cycle dose(s), the at least one non-pharmacologic modality, determination of the relevancy of the at least one surrogate markers and determination of the relevancy of the at least one non-pharmacologic modality.

Yet another embodiment of the present invention advantageously provides a program product or software to revise a cycle dose of at least one agent in a therapy for a patient receiving such a therapy. This program product, for example, includes a first receiver to accept as a first input the patient's cycle dose(s) of the at least one agent, a second receiver to accept as a second input determination of the relevancy of at least one surrogate marker indicating a pharmacologic response of the patient, and a calculator to calculate a revised cycle dose of the at least one agent.

Yet another embodiment of the present invention advantageously provides a program product or software to revise a cycle dose of at least one agent in a therapy for a patient receiving such a therapy. This program product advantageously includes a first receiver to accept as a first input the patient's cycle dose(s) of the at least one agent, a second receiver to accept as a second input at least one non-pharmacologic modality received by the patient, a third receiver to accept as a third input determination of the relevancy of at least one surrogate marker indicating a pharmacologic response of the patient, a fourth receiver to accept as a fourth input determination of the relevancy of the at least one non-pharmacologic modality indicating achievement of benefit, and a calculator to calculate a revised cycle dose of the at least one agent as a function of the first input cycle dose(s), the at least one non-pharmacologic modality, determination of the relevancy of the at least one surrogate markers and determination of the relevancy of the at least one non-pharmacologic modality.

Advantageously, embodiments of the methods, systems and program products of the present invention each focuses on a cycle dose, rather than a current dose as known in the art. Cycle dose is a summative or cumulative dose administered for a time period during which the maximum therapeutic effect of a single or multiple agent(s) is realized. Applicants have recognized that it is the agent's total amount over a time period based upon the specifics of the agent and the disorder is more accurate, appropriate and effective. The present invention considers doses administered during a selected period of time, which better accommodate the variability of compliance and the time needed for the agent to demonstrate its efficacy or toxicity. The cycle dose is also based upon how a prescriber actually practices, thinks and acts out the process.

Still advantageously, embodiments of the methods, systems and program products of the present invention each can analyze two or more surrogate markers concurrently rather than one marker at a time. This multi-marker analysis provides a real-time measurement of the cumulative affect of one or more pharmacologic agents and one or more non-pharmacologic modalities. Further advantageously, embodiments of the methods, systems and program products of the present invention can provide more accurate information on efficacy of drugs that have been shelved for a long time, which would help pharmaceutical companies to reclaim the drugs. Additionally, these embodiments are non-invasive, precise and sensitive.

Although embodiments of the present invention have been described in the context of a fully functional system 400, apparatus 410, 420¹, and program product 442, 442¹ of the present invention and/or aspects thereof are capable of being distributed in the form of computer readable medium, media, or means of instructions in a variety of forms for execution on one or more processors such as used in association with various types of computers, including, but not limited to, laptops, personal digital assistants, server computers, administration computers, and various other hardwired, software, and/or firmware as understood by those skilled in the art. Also, these embodiments of the present invention can also apply regardless of the particular type of signal bearing media or means used to actually carry out the processing, distributing, or dosing as described herein. Examples or computer readable media or means include: nonvolatile, hard-coded type media such as read only memories (RAMs), erasable, electrically programmable read only memories (EEPROMs), including non-volatile types, recordable and writable media such as CDs, DVDs, floppy disks, hard disk drives, and transmission type media such as digital and analog communication links.

In the drawings and specification, there have been disclosed embodiments of the invention and, although specific terms are employed, they are used in a generic and descriptive sense only and not for the purpose of limitation, the scope of the invention being set forth in the following claims. The invention has been described in considerable detail with specific reference to these illustrated embodiments. It will be apparent, however, that various modifications and changes can be made within the spirit and scope of the invention as described in the foregoing specification.

1. A method for revising a cycle dose of at least one agent in a therapy for a patient receiving such a therapy, the method comprising the steps of:
accepting as a first input the patient's cycle dose of the at least one agent;
accepting as a second input determination of the relevancy of at least one surrogate marker indicating a pharmacologic response of the patient; and
determining a revised cycle dose of the at least one agent as a function of the first input cycle dose and second input determination of the relevancy of the at least one surrogate marker.

2. A method as defined in claim 1, wherein the cycle dose comprises a total amount of the at least one agent over a selected period of time.

3. A method as defined in claim 1, wherein the therapy includes a single and multi-agent therapy.

4. A method as defined in claim 1, further comprising the steps of:
accepting as a third input at least one non-pharmacologic modality the patient may be receiving; and
accepting as a fourth input determination of the relevancy of the at least one non-pharmacologic modality indicating achievement of a benefit.

5. A method as defined in claim 4, wherein the revised cycle dose of the at least one agent is a function of the first input cycle dose, the at least one non-pharmacologic modality, determination of the relevancy of the at least one surrogate marker and determination of the relevancy of the at least one non-pharmacologic modality.

6. A method as defined in claim 1, wherein two or more surrogate markers are analyzed concurrently.

7. A method for revising a cycle dose of at least one agent in a therapy for a patient receiving such a therapy, the method comprising the steps of:
accepting as a first input the patient's cycle dose of the at least one agent;
accepting as a second input at least one non-pharmacologic modality received by the patient;
accepting as a third input determination of the relevancy of at least one surrogate marker indicating a pharmacologic response of the patient;
accepting as a fourth input determination of the relevancy of the at least one non-pharmacologic modality indicating achievement of maximum benefit; and
determining a revised cycle dose of the at least one agent responsive to the cumulative effect of the at least one agent and the at least one non-pharmacologic modality to be achieved by the therapy and non-pharmacologic intervention, the revised cycle dose being a function of the first input cycle dose, the at least one non-pharmacologic modality, determination of the relevancy of the at least one surrogate marker and determination of the relevancy of the at least one non-pharmacologic modality.

8. A method as defined in claim 7, wherein two or more surrogate markers are analyzed concurrently.

9. A system to calculate a revised cycle dose of at least one agent in a therapy for a patient receiving such a therapy, the system comprising:
means for accepting as a first input the patient's cycle dose of the at least one agent;
means for accepting as a second input determination of the relevancy of at least one surrogate marker indicating a pharmacologic response of the patient; and
means responsive to the first input and second input for calculating a revised cycle dose of the at least one agent.

10. A system as defined in claim 9, further comprising:
means for accepting as a third input at least one non-pharmacologic modality received by the patient; and
means for accepting as a fourth input determination of the relevancy of the at least one non-pharmacologic modality indicating achievement of a benefit.

11. A system as defined in claim 10, wherein the revised cycle dose of the at least one agent is a function of the first input cycle dose, the at least one non-pharmacologic modality, determination of the relevancy of the at least one surrogate marker and determination of the relevancy of the at least one non-pharmacologic modality.

12. A system as defined in claim 11, wherein two or more surrogate markers are analyzed concurrently.

13. A system to calculate a revised cycle dose of at least one agent in a therapy for a patient receiving such a therapy, the system comprising:
means for accepting as a first input the patient's cycle dose of the at least one agent;
means for accepting as a second input at least one non-pharmacologic modality received by the patient;
means for accepting as a third input determination of the relevancy of at least one surrogate marker indicating a pharmacologic response of the patient;
means for accepting as a fourth input determination of the relevancy of the at least one non-pharmacologic modality indicating achievement of maximum benefit; and
means responsive to the first, second, third and fourth inputs for calculating a revised cycle dose of the at least one agent as a function of the first input cycle dose, the at least one non-pharmacologic modality, determination of the relevancy of the at least one surrogate marker and determination of the relevancy of the at least one non-pharmacologic modality.

14. A system as defined in claim 13, wherein two or more surrogate markers are analyzed concurrently.

15. A program product stored on a tangible computer memory medium to calculate a revised cycle dose of at least one agent in a therapy for a patient receiving such a therapy, the program product comprising:
means for accepting as a first input the patient's cycle dose of the at least one agent;
means for accepting as a second input determination of the relevancy of at least one surrogate marker indicating a pharmacologic response of the patient; and
means responsive to the effect of the at least one agent to be achieved by the therapy for calculating a revised cycle dose of the at least one agent as a function of the first input cycle dose and second input determination of the relevancy of the at least one surrogate marker.

16. A program product as defined in claim 15, wherein two or more surrogate markers are analyzed concurrently.

17. A program product stored on a tangible computer memory medium to calculate a revised cycle dose of at least one agent in a therapy for a patient receiving such a therapy, the program product comprising:
means for accepting as a first input the patient's cycle dose of the at least one agent;
means for accepting as a second input at least one non-pharmacologic modality received by the patient;
means for accepting as a third input determination of the relevancy of at least one surrogate marker indicating a pharmacologic response of the patient;
means for accepting as a fourth input determination of the relevancy of the at least one non-pharmacologic modality indicating achievement of maximum benefit; and
means responsive to the cumulative effect of the at least one agent and the at least one non-pharmacologic modality to be achieved by the therapy and non-pharmacologic intervention for calculating a revised cycle dose of the at least one agent as a function of the first input cycle dose(s), the at least one non-pharmacologic modality, determination of the relevancy of the at least one surrogate marker and determination of the relevancy of the at least one non-pharmacologic modality.

18. A program product as defined in claim 17, wherein two or more surrogate markers are analyzed concurrently.

19. A program product stored on a tangible computer memory medium to calculate a revised cycle dose of at least one agent in a therapy for a patient receiving such a therapy, the program product comprising:
a first receiver to accept as a first input the patient's cycle dose(s) of the at least one agent;
a second receiver to accept as a second input determination of the relevancy of at least one surrogate marker indicating a pharmacologic response of the patient; and
a calculator to calculate a revised cycle dose of the at least one agent as a function of the first input cycle dose and second input determination of the relevancy of the at least one surrogate marker in response to the effect of the at least one agent to be achieved by the therapy.

20. A program product as defined in claim 19, wherein two or more surrogate markers are analyzed concurrently.

21. A program product stored on a tangible computer memory medium to calculate a revised cycle dose of at least one agent in a therapy for a patient receiving such a therapy, the program product comprising:
a first receiver to accept as a first input the patient's cycle dose of the at least one agent;
a second receiver to accept as a second input at least one non-pharmacologic modality received by the patient;
a third receiver to accept as a third input determination of the relevancy of at least one surrogate marker indicating a pharmacologic response of the patient;
a fourth receiver to accept as a fourth input determination of the relevancy of the at least one non-pharmacologic modality indicating achievement of maximum benefit; and
a calculator to calculate a revised cycle dose of the at least one agent as a function of the first input cycle dose, the at least one non-pharmacologic modality, determination of the relevancy of the at least one surrogate marker and determination of the relevancy of the at least one non-pharmacologic modality in response to the cumulative effect of the at least one agent and the at least one non-pharmacologic modality to be achieved by the therapy and non-pharmacologic intervention.

22. A program product as defined in claim 21, wherein two or more surrogate markers are analyzed concurrently.
